(19) United States
(12) Patent Application Publication
(10) Pub. No.: US 2010/0158863 A1
(43) Pub. Date: Jun. 24, 2010

(54) TRIAZOLOANILINOPYRIMIDINE DERIVATIVES FOR USE AS ANTIVIRAL AGENTS

(75) Inventors: Neil Mathews, London (GB); Alexander James Floyd Thomas, London (GB); Keith Charles Spencer, London (GB); Nathalie Tiberghien, London (GB); Christopher John Pilkington, London (GB); Lyn Jennens, London (GB); Surinder Chana, London (GB); Ian John Fraser, London (GB)

Correspondence Address: ASTRAZENECA R&D BOSTON, 35 GATEHOUSE DRIVE, WALTHAM, MA, 02451-1215, US

(73) Assignee: ARROW THERAPEUTICS LIMITED, London, EN (GB)

(21) Appl. No.: 12/160,506

(22) PCT Filed: Jan. 11, 2007
(86) PCT No.: PCT/GB2007/000065

§ 371 Date: Jul. 18, 2008

(30) Foreign Application Priority Data
Jan. 11, 2006 (GB) 0600510.2
Jan. 19, 2006 (GB) 0612116.4

(51) Int. Cl. A61K 31/517; C07D 403/12; C07D 413/14; A61K 31/5377; A61P 31/14; A61K 38/21; A61K 31/7056

(52) U.S. Cl. 424/85.4; 544/284; 514/266.23; 544/119; 514/234.5; 514/266.21; 514/252.17; 514/43

(57) ABSTRACT

A quinazoline derivative of formula (I), or a pharmaceutically acceptable salt thereof: formula (I) for use in treatment of prevention of a flaviviridae infection.

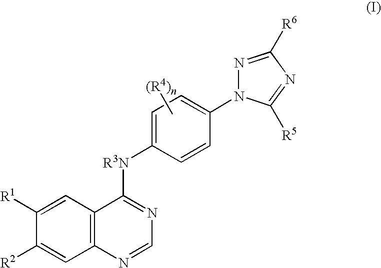

The present invention relates to a series of quinazoline derivatives which are useful in treating or preventing a flaviviridae infection.

Viruses of the family flaviviridae are small, icosahedral, enveloped viruses that contain a positive-sense RNA genome. The family consists of three genera, flavivirus, pestivirus and hepacivirus.

Many of the flaviviridae viruses are important human pathogens. Indeed, the hepacivirus genus includes the hepatitis C virus. However, there exists, as yet, no effective and safe treatment for flaviviridae infections.

WO 98/02434 discloses quinazolines as protein tyrosine kinase inhibitors. None of the compounds specifically disclosed in that document carry a triazolylaniline group at the 6-position.

It has now surprisingly been found that the quinazoline derivatives of the formula (I) are active in inhibiting replication of flaviviridae viruses and are therefore effective in treating or preventing a flaviviridae infection. The present invention therefore provides a quinazoline derivative of formula (I), or a pharmaceutically acceptable salt thereof,

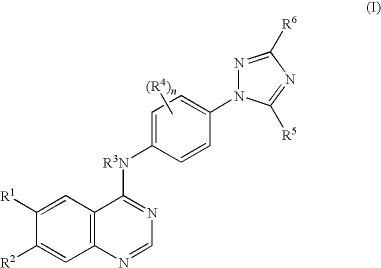

wherein:
R¹ represents halogen, C₁₋₆ alkyl, C₁₋₄ haloalkyl, C₁₋₆ alkoxy, C₁₋₄ haloalkoxy or a moiety -A, -A-A′, -A-Het-A′, -A-L-A′, -A-Het-L-A′, -A-L-Het-A′, -A-Het-L-Het′-A′ or -A-Het-L-Het′-L′;
R² represents hydrogen, halogen, C₁₋₄ alkyl, C₁₋₄ alkoxy, C₁₋₄ haloalkyl, C₁₋₄ haloalkoxy or a moiety -Het-A′, -Het-L-A′, -Het-L-Het′-A′ or -Het-L-Het′-L′;
R³ represents hydrogen or C₁₋₆ alkyl;
each R⁴ is the same or different and represents halogen, C₁₋₄ alkyl, C₁₋₄ alkoxy or C₁₋₄ haloalkyl;
R⁵ represents hydrogen, C₁₋₄ alkyl, C₁₋₄ haloalkyl, C₁₋₄ alkoxy, C₁₋₄ aminoalkyl or a moiety -Het-L-Het′-L′;
R⁶ represents hydrogen, C₁₋₄ alkyl, C₁₋₄ haloalkyl, C₁₋₄ alkoxy, C₁₋₄ aminoalkyl or a moiety -Het-L-Het′-L′, —CO—X or -L-X where X represents —OR′, —NR′R″ or a 5- to 10-membered heteroaryl or heterocyclyl group, wherein R′ and R″ are independently hydrogen or C₁₋₄ alkyl;
n is zero, 1 or 2;
each A and A′ are the same or different and represent a phenyl, 5- to 10-membered heteroaryl, 5- to 10-membered heterocyclyl or C₃₋₆ carbocyclyl group which is optionally fused to a further phenyl, 5- to 10-membered heteroaryl, 5- to 10-membered heterocyclyl or C₃₋₆ carbocyclyl group;
each Het and Het′ is the same or different and represents —O—, —S— or —NR′— where R′ is hydrogen or C₁₋₄ alkyl;
each L is the same or different and represents C₁₄ allylene; and
each L′ is the same or different and represents hydrogen or a C₁₋₄ alkyl, C₁₋₄ haloalkyl, C₁₋₄ hydroxyallyl or C₁₄ aminoalkyl group,

the phenyl, heteroaryl, heterocyclyl and carbocyclyl moieties in R₁, R₂ and R₆ being unsubstituted or substituted by 1, 2 or 3 unsubstituted substituents which are the same or different and are selected from halogen atoms and C₁₋₄ alkyl, C₁₋₄ alkoxy, C₁₋₄ haloalkyl, C₁₋₄ haloalkoxy, hydroxy, cyano, nitro and —NR′R″, wherein each R′ and R″ is the same or different and represents hydrogen or C₁₋₄ alkyl.

In another embodiment, the present invention provides a quinazoline derivative of the formula (I), as defined above, or a pharmaceutically acceptable salt thereof, wherein:
R¹ represents halogen, C₁₋₆ alkyl, C₁₋₄ haloalkyl, C₁₋₆ alkoxy, C₁₋₄ haloalkoxy or a moiety -A, -A-A′, -A-Het-A′, -A-L-A′, -A-Het-L-A′, -A-L-Het-A′, -A-Het-L-Het′-A′ or -A-Het-L-Het′-L′;
R² represents hydrogen, halogen, C₁₋₄ alkyl, C₁₋₄ alkoxy, C₁₋₄ haloalkyl, C₁₋₄ haloalkoxy or a moiety -Het-A′, -Het-L-A′, -Het-L-Het′-A′ or -Het-L-Het′-L′;
R³ represents hydrogen or C₁₋₆ alkyl;
each R⁴ is the same or different and represents halogen, C₁₋₄ alkyl, C₁₋₄ alkoxy or C₁₋₄ haloalkyl;
R⁵ represents hydrogen, C₁₋₄ alkyl, C₁₋₄ haloalkyl, C₁₋₄ alkoxy, C₁₋₄ aminoalkyl or a moiety -Het-L-Het′-L′;
R⁶ represents hydrogen, C₁₋₄ alkyl, C₁₋₄ haloalkyl, C₁₋₄ alkoxy, C₁₋₄ aminoalkyl or a moiety -Het-L-Het′-L′, —CO—X or -L-X where X represents —OR′, —NR′R″ or a 5- to 10-membered heteroaryl or heterocyclyl group, wherein R′ and R″ are independently hydrogen or C₁₋₄ alkyl;
n is zero, 1 or 2;
each A and A′ are the same or different and represent dphenyl, 5- to 10-membered heteroaryl, 5- to 10-membered heterocyclyl or C₃₋₆ carbocyclyl group which is optionally fused to a further phenyl, 5- to 10-membered heteroaryl, 5- to 10-membered heterocyclyl or C₃₋₆ carbocyclyl group;
each Het and Het′ is the same or different and represents —O—, —S— or —NR′— where R′ is hydrogen or C₁₄ allyl;
each L is the same or different and represents C₁₋₄ alkylene; and
each L′ is the same or different and represents hydrogen or a C₁₋₄ alkyl, C₁₋₄ haloalkyl or C₁₋₄ hydroxyalkyl group,

the phenyl, heteroaryl, heterocyclyl and carbocyclyl moieties in A and A′ being unsubstituted or substituted by 1, 2 or 3 unsubstituted substituents which are the same or different and are selected from halogen atoms and C₁₋₄ alkyl, C₁₋₄ alkoxy, C₁₋₄ haloalkyl, C₁₋₄ haloalkoxy, hydroxy, cyano, nitro and —NR′R″, wherein each R′ and R″ is the same or different and represents hydrogen or C₁₋₄ alkyl.

As used herein, a C₁₋₆ alkyl group or moiety is a linear or branched alkyl group or moiety containing from 1 to 6 carbon atoms, for example 1 to 4 carbon atoms. Examples of C₁₋₆ alkyl groups and moieties include C₁₋₄ alkyl groups and moieties such as include methyl, ethyl, n-propyl, i-propyl, n-butyl, i-butyl and t-butyl. For the avoidance of doubt, where two alkyl moieties are present in a group, the alkyl moieties may be the same or different.

As used herein, a C₁₋₄ alkylene group or moiety is a linear or branched alkylene group or moiety. Examples include methylene, n-ethylene and n-propylene groups and moieties.

As used herein, a halogen is typically chlorine, fluorine, bromine or iodine and is preferably chlorine, bromine or fluorine, more preferably chlorine, fluorine or iodine.

As used herein, a C₁₋₆ alkoxy group is typically a said C₁₋₆ alkyl group attached to an oxygen atom. A haloalkyl or haloalkoxy group is typically a said alkyl or alkoxy group substituted by one or more said halogen atoms. Typically, it is substituted by 1, 2 or 3 said halogen atoms. Preferred haloalkoxy groups include alkoxy groups substituted by one or two chlorine atoms, more preferably by one chlorine atom. Particularly preferred haloalkyl groups include —O—(CH₂)₃—Cl. Other preferred haloalkyl and haloalkoxy groups include perhaloalkyl and perhaloalkoxy groups such as —CX₃ and —OCX₃ wherein X is a said halogen atom, for example chlorine and fluorine. Particularly preferred haloalkyl groups are —CF₃ and —CCl₃.

As used herein, a C₁₋₄ hydroxyalkyl group is a C₁₋₄ alkyl group substituted by one or more hydroxy groups. Typically, it is substituted by one, two or three hydroxy groups. Preferably, it is substituted by a single hydroxy group. A preferred hydroxyalkyl group is —(CH₂)₂—OH.

As used herein, a C₁₋₄ aminoalkyl group is a C₁₋₄ alkyl group substituted by one or more —NR′R″ groups wherein each R′ and R″ is the same or different and represents hydrogen or C₁₋₄ alkyl. Typically it is substituted by one, two or three —NR′R″ groups wherein each R′ and R″ is the same or different and represents hydrogen or C₁₋₄ alkyl. Preferably each R′ and R″ is the same or different and represents hydrogen or C₁₋₂ alkyl, more preferably hydrogen or methyl. Preferably the C₁₋₄ alkyl group is substituted by a single —NR′R″ group as defined above. More preferably, the C₁₋₄ alkyl group is substituted by a single group —N(CH₃)₂.

As used herein, a 5- to 10-membered heteroaryl group or moiety is a monocyclic 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl and pyrazolyl groups. Furanyl, thienyl, pyridyl and pyrimidyl groups are preferred.

When A or A′ is a 5- to 10-membered heteroaryl moiety fused to a phenyl, 5- to 10-membered heteroaryl, 5- to 10-membered heterocyclyl or C₃₋₆ carbocyclyl group, it is typically fused to a phenyl, 5- to 6-membered heteroaryl or 5- to 6-membered heterocyclyl ring. More preferably, it is fused to a phenyl ring.

When A or A′ is a phenyl group fused to a further phenyl, 5- to 10-membered heteroaryl, 5- to 10-membered heterocyclyl or C₃₋₆ carbocyclyl group, it is preferably fused to a phenyl, 5- to 6-membered heteroaryl or 5- to 6-membered heterocyclyl ring. More preferably, it is fused to a 5- to 6-membered heteroaryl or heterocyclyl ring. Most preferably, it is fused to a 1,4-dioxacyclohexane ring.

As used herein, a 5- to 10-membered heterocyclyl group or moiety is a monocyclic non-aromatic, saturated or unsaturated C₅-C₁₀ carbocyclic ring in which one or more, for example 1, 2 or 3, of the carbon atoms are replaced with a moiety selected from N, O, S, S(O) and S(O)₂. Typically, it is a 5- to 6-membered ring.

Suitable heterocyclyl groups and moieties include pyrazolidinyl, piperidyl, piperazinyl, thiomorpholinyl, S-oxo-thiomorpholinyl, S,S-dioxo-thiomorpholinyl, morpholinyl, pyrrolidinyl, pyrrolinyl, imidazolidinyl, imidazolinyl, 1,3-dioxolanyl, 1,4-dioxolanyl, 1,2-dioxacyclohexyl, 1,3-dioxacyclohexyl, 1,4-dioxacyclohexyl and pyrazolinyl groups and moieties. Preferred heterocyclyl groups are piperazinyl, pyrrolidinyl, morpholinyl and 1,4-dioxacyclohexane groups, in particular morpholinyl and 1,4-dioxacyclohexane groups.

When A or A′ is a said 5- to 10-membered heterocyclyl moiety fused to a further phenyl, 5- to 10-membered heteroaryl, 5- to 10-membered heterocyclyl or C₃₋₆ carbocyclyl group it is preferably fused to a phenyl, 5- to 6-membered heteroaryl or 5- to 6-membered heterocyclyl ring. More preferably, it is fused to a phenyl ring.

For the avoidance of doubt, although the above definitions of heteroaryl and heterocyclyl groups refer to an “N” moiety which can be present in the ring, as will be evident to a skilled chemist the N atom will be protonated (or will carry a substituent as defined above) if it is attached to each of the adjacent ring atoms via a single bond.

As used herein, a C₃₋₆ carbocyclic moiety is a monocyclic non-aromatic saturated or unsaturated hydrocarbon ring having from 3 to 6 carbon atoms. Preferably it is a saturated hydrocarbon ring (i.e. a cycloalkyl moiety) having from 3 to 6 carbon atoms. Examples include cyclopropyl, cyclobutyl, cyclopentyl and cyclohexyl.

When A or A′ is a said C₃₋₆ carbocyclyl group fused to a further phenyl, 5- to 10-membered heteroaryl, 5- to 10-membered heterocyclyl or C₃₋₆ carbocyclyl group, it is preferably fused to a phenyl, 5- to 6-membered heteroaryl or 5- to 6-membered heterocyclyl ring.

When the said phenyl, heteroaryl, heterocyclyl and carbocyclyl moieties are substituted by two or three substituents, it is preferred that not more than one substituent is selected from cyano and nitro.

Typically, the phenyl, heteroaryl, heterocyclyl and carbocyclyl moieties in R¹, R² and R⁶ are unsubstituted or substituted by 1, 2 or 3 unsubstituted substituents which are the same or different and are selected from halogen, C₁₋₄ alkyl, C₁₋₄ alkoxy, C₁₋₄ haloalkyl, C₁₋₄ haloalkoxy, hydroxy and —NR′R″ wherein each R′ and R″ is the same or different and represents hydrogen or C₁₋₄ alkyl. More typically, in this embodiment, the cyclic group in R⁶ is unsubstituted and the phenyl, heteroaryl, heterocyclyl and carbocyclyl moieties in the A and A′ groups are unsubstituted or substituted by 1, 2 or 3 unsubstituted substituents which are the same or different and are selected from halogen, C₁₋₄ alkyl, C₁₋₄ alkoxy, C₁₋₄ haloalkyl, C₁₋₄ haloalkoxy, hydroxy and —NR′R″ wherein each R′ and R″ is the same or different and represents hydrogen or C₁₋₄ alkyl.

Preferably the phenyl, heteroaryl, heterocyclyl and carbocyclyl moieties in R¹, R² and R⁶ are unsubstituted or substituted with 1, 2 or 3 unsubstituted substituents which are the same or different and are selected from halogen, hydroxy, C₁₋₄ alkyl, C₁₋₄ alkoxy, C₁₋₄ haloalkyl and C₁₋₄ haloalkoxy. More typically, in this preferred embodiment, the cyclic group in R⁶ is unsubstituted and the phenyl, heteroaryl, heterocyclyl and carbocyclyl moieties in the A and A′ groups are unsubstituted or substituted with 1, 2 or 3 unsubstituted substituents which are the same or different and are selected from halogen, C₁₋₄ alkyl, C₁₋₄ alkoxy, C₁₋₄ haloalkyl and C₁₋₄ haloalkoxy.

More preferably, the phenyl, heteroaryl, heterocyclyl and carbocyclyl moieties in R¹, R² and R⁶ are unsubstituted or substituted with 1 or 2 unsubstituted substituents which are the same or different and are selected from halogen, for example chlorine and fluorine, hydroxy, C₁₋₄ alkyl, for example methyl and C₁₋₄ alkoxy. More typically, in this embodiment, the cyclic group in R⁶ is unsubstituted and the phenyl, heteroaryl, heterocyclyl and carbocyclyl moieties in the A and A′ groups are substituted or substituted with 1 or 2 unsubstituted substituents which are the same or different and are selected from halogen, for example chlorine and fluorine, C₁₋₄ alkyl, for example methyl and C₁₋₂ alkoxy.

Typically, each A is the same or different and is a phenyl, 5- to 6-membered heteroaryl or 5- to 6-membered heterocyclyl group which is optionally fused to a further phenyl, 5- to 6-membered heteroaryl, 5- to 6-membered heterocyclyl or C₃₋₆ carbocyclyl group. Preferably each A is the same or different and is a phenyl or a 5- to 6-membered heteroaryl group which is optionally fused to a further phenyl, 5- to 6-membered heteroaryl or 5- to 6-membered heterocyclyl group. More preferably each A is the same or different and is a non-fused phenyl or 5- to 6-membered heteroaryl group or is a phenyl ring fused to a further phenyl, 5- to 6-membered heteroaryl or 5- to 6-membered heterocyclyl group. When A is a 5- to 6-membered heteroaryl group it is preferably a pyrimidinyl, furanyl or thienyl group. When A is a fused group it is preferably a phenyl group fused to a 5- to 6-membered heterocyclyl group, most preferably a 1,4-dioxacyclohexyl group.

Typically A is unsubstituted or substituted with 1 or 2 unsubstituted substituents which are the same or different and are selected from halogen, hydroxy, C₁₋₄ alkyl and C₁₋₄ alkoxy. More typically, these substituents are selected from halogen, C₁₋₄ alkyl and C₁₋₂ alkoxy groups.

Typically each A′ is the same or different and is a phenyl, a 5- to 6-membered heteroaryl or a 5- to 6-membered heterocyclyl group and is optionally fused to a further phenyl, a 5- to 6-membered heteroaryl, a 5- to 6-membered heterocyclyl or a C₃₋₆ carbocyclyl group. More typically A′ is a non-fused phenyl, 5- to 6-membered heteroaryl or 5- to 6-membered heterocyclyl. Most preferably A′ is a non-fused 5- to 6-membered heteroaryl or heterocyclyl group, for example pyridyl, pyrrolidinyl and morpholinyl, in particular pyridyl and morpholinyl.

Typically A′ is unsubstituted or substituted with 1 or 2 unsubstituted substituents which are the same or different and are selected from halogen, C₁₋₄ alkyl and C₁₋₂ alkoxy. Preferably A′ is unsubstituted.

Typically Het is —O—, —S— or —NR′— where R′ is hydrogen or C₁₋₂ alkyl. More preferably Het is —O— or —NR′— where R′ is hydrogen or C₁₋₂ alkyl. More preferably Het is —O—.

Typically Het′ is —O—, —S— or —NR′— where R′ is hydrogen or C₁₋₂ alkyl. More preferably Het′ is —O— or —NR′— where R′ is hydrogen or C₁₋₂ alkyl. When Het′ is —NR′—, preferably R′ is methyl.

Typically each L is the same or different and represents C₁₋₄ alkylene. More preferably each L is independently selected from methylene, n-ethylene or n-propylene.

Preferably, each L′ is the same or different and represents hydrogen or a C₁₋₂ alkyl, C₁₋₂ haloalkyl, C₁₋₂ hydroxyalkyl or C₁₋₂ aminoalkyl group. Typically, these preferred L′ moieties are selected from hydrogen and C₁₋₂ alkyl, C₁₋₂ haloalkyl and C₁₋₂ hydroxyalkyl groups.

More preferably, each L′ is the same or different and represents hydrogen, C₁₋₂ alkyl, C₁₋₂ aminoalkyl or C₁₋₂ hydroxyalkyl. Typically, those more preferred L′ moieties are selected from hydrogen, C₁₋₂ alkyl and C₁₋₂ hydroxyalkyl.

When L′ is C₁₋₂ alkyl it is preferably a methyl group. When L′ is C₁₋₂ hydroxyalkyl it is preferably —(CH₂)₂OH. When L′ is C₁₋₂ aminoalkyl it is preferably —(CH₂)₂—N(CH₃)₂.

When R¹ is -A, typically R¹ is a phenyl or a 5- to 6-membered heteroaryl group and is optionally fused to a further phenyl, 5- to 6-membered heteroaryl or 5- to 6-membered heterocyclyl group. More preferably R¹ is phenyl, 5- to 6-membered heteroaryl or a phenyl fused to a further phenyl, 5- to 6-membered heteroaryl or 5- to 6-membered heterocyclyl group. More preferably R¹ is phenyl, pyrimidinyl, furanyl or thienyl or is a phenyl group fused to a 1,4-dioxacyclohexyl group.

When R¹ is -A, typically R¹ is unsubstituted or substituted with 1 or 2 unsubstituted substituents which are the same or different and are selected from halogen, hydroxy, C₁₋₄ alkyl and C₁₋₄ alkoxy. Typically, those preferred substituents are selected from halogen, C₁₋₄ alkyl and C₁₋₂ alkoxy. Preferred halogen substituents include fluorine. Preferred C₁₋₄ alkyl substituents include methyl. Preferred C₁₋₂ alkoxy substituents include methoxy and ethoxy.

When R¹ is -A-A′, R¹ is typically a moiety-phenyl-A′ wherein A′ is a non-fused 5- to 6-membered heterocyclyl group. More preferably A′ is morpholinyl.

When R¹ is -A-Het-L-Het′-L′, R¹ is typically a moiety-phenyl-O-L-Het′-L′. L is typically n-ethylene. Het′ is typically —O— or —NR′— wherein R′ is hydrogen or C₁₋₂ alkyl. More preferably Het′ is —O— or —NMe—. L′ is typically methyl or C₁₋₂ aminoalkyl, for example —(CH₂)₂—N(CH₃)₂. Preferably, L′ is methyl.

When R¹ is -A-Het-L-A′, R¹ is typically a moiety-phenyl-O-L-A′. L is typically methylene or n-propylene, preferably methylene. A′ is typically a 5- to 6-membered heteroaryl or a 5- to 6-membered heterocyclyl group. Typically A′ is non-fused. More preferably A′ is a non-fused 5- to 6-membered heteroaryl or heterocyclyl group, more preferably an unsubstituted pyridyl, pyrrolidinyl or morpholino group. More typically, in this embodiment these preferred A′ moieties are selected from non-fused 5- to 6-membered heteroaryl groups, preferably unsubstituted pyridyl groups.

When R¹ is -A-A′, -A-Het-A′, -A-L-A′, -A-Het-L-A′, -A-L-Het-A′, -A-Het-L-Het′-A′ or -A-Het-L-Het′-L′, typically A is unsubstituted or substituted with 1 or 2 unsubstituted substituents which are the same or different and are selected from halogen, C₁₋₄ alkyl and C₁₋₂ alkoxy. More preferably A is unsubstituted or is substituted with a halogen atom, in particular fluorine. Most preferably, A is unsubstituted. Similarly, when R¹ is -A-A′, -A-Het-A′, -A-L-A′, -A-Het-L-A′, -A-L-Het-A′, -A-Het-L-Het′-A′ or -A-Het-L-Het′-L′, typically A′ is unsubstituted or substituted with 1 or 2 unsubstituted substituents which are the same or different and are selected from halogen, C₁₋₄ alkyl and C₁₋₂ alkoxy. More preferably A′ is unsubstituted.

When R¹ is halogen, typically it is a chlorine, bromine or iodine atom, more preferably a bromine or iodine atom, most preferably an iodine atom.

When R¹ is C₁₋₆ alkyl, typically it is a C₁₋₄ alkyl group, for example a tertiary-butyl group.

When R¹ is C₁₋₄ alkoxy group, typically it is a C₁₋₂ alkoxy group, more preferably a methoxy group.

Preferably R¹ represents halogen, C₁₋₄ alkyl, C₁₋₂ alkoxy or a moiety -A, -A-A′, -A-Het-A′, -A-L-A′, -A-Het-L-A′, -A-L-Het-A′, -A-Het-L-Het′-A′ or -A-Het-L-Het′-L′ where A, A′, Het, Het′, L and L′ are as defined earlier.

More preferably R¹ represents halogen, for example bromine and iodine, C₁₋₄ alkyl, C₁₋₂ alkoxy, -A, -A-A′, -A-Het-L-A′ or -A-Het-L-Het′-L′. More preferably R¹ represents halogen, for example bromine and iodine, C₁₋₄ alkyl, C₁₋₂ alkoxy, -A, -A-A′, -A-O-L-A′ or -A-O-L-Het′-L′.

In a further embodiment of the invention, R₁ represents -A, -A-A′, -A-Het-A′, -A-L-A′, -A-Het-L-A′, -A-L-Het-A′, -A-Het-L-Het′-A′ or -A-Het-L-Het′-L′, wherein A is a phenyl group which is optionally fused to a further phenyl, 5- to 10-membered heteroaryl, 5- to 10-membered heterocyclyl or C₃₋₆ carbocyclyl group, and A′, Het, L, Het′ and L′ are as defined above.

When R² is C₁₋₄ haloalkoxy it preferably substituted by 1 or 2 halogen atoms. Preferred halogen atoms are chlorine atoms. When R² is C₁₋₄ haloalkoxy preferably it is —O—(CH₂)₃—Cl.

When R² represents -Het-L-A′, R² is typically a moiety —O-L-A′. L is preferably n-propylene. A′ is typically a non-fused 5- to 6-membered heterocyclyl group, for example morpholine. When R² represents -Het-L-A′ preferably A′ is unsubstituted.

When R² represents -Het-L-Het′-L′, Het′ is preferably —O— or —NR′— wherein R′ is hydrogen or C₁₋₂ alkyl. More preferably Het′ is —NMe—. When R² represents -Het-L-Het′-L′, Het is preferably —O—. When R² represents -Het-L-Het′-L′, L is preferably n-propylene. When R² represents -Het-L-Het′-L′, L′ is preferably —(CH₂)₂—OH.

Preferably R² represents hydrogen, halogen, C₁₋₄ alkyl, C₁₋₄ alkoxy, C₁₋₄ haloalkyl, C₁₋₄ haloalkoxy or a moiety -Het-L-A′ or -Het-L-Het′-L′ where Het, Het′, L, L′ and A′ are as defined earlier. More preferably R² represents hydrogen, C₁₋₄ alkoxy, C₁₋₄ haloalkoxy or a moiety -Het-L-A′ or -Het-L-Het′-L′ where Het, Het′, L, L′ and A′ are as defined earlier.

More preferably R² represents hydrogen, C₁₋₄ haloalkoxy or a moiety -Het-L-A′ or -Het-L-Het′-L′ where Het, Het′, L, L′ and A′ are as defined earlier.

Preferably R³ is hydrogen or C₁₋₂ alkyl. More preferably R³ is hydrogen.

Preferably each R⁴ is the same or different and represents halogen or C₁₋₄ alkyl. More preferably, each R₄ is the same or different and represents C₁₋₂ alkyl, in particular methyl. Preferably n is zero or 1. More preferably n is zero.

Preferably R⁵ is hydrogen, C₁₋₄ alkyl, C₁₋₄ haloalkyl, C₁₋₄ alkoxy or C₁₋₄ aminoalkyl. More preferably R⁵ is hydrogen or C₁₋₄ alkyl, more preferably hydrogen or methyl, most preferably hydrogen.

Typically, X in the R⁶ moiety represents —OR′, —NR′R″ or a 5- to 6-membered heteroaryl or heterocyclyl group, wherein R′ and R″ are as defined above. Typically, said heteroaryl or heterocyclyl group is unsubstituted or substituted with one or two unsubstituted substituents selected from halogen, C₁₋₄ alkyl, C₁₋₄ haloalkyl and C₁₋₄ alkoxy. Preferably, said heteroaryl or heterocyclyl group is a heterocyclyl group, in particular a piperazinyl group, which is unsubstituted or substituted by a C₁₋₂ alkyl group. Typically, R′ and R″ in the moiety —NR′R″ are the same or different and represent hydrogen or C₁₋₂ alkyl.

Typically, R₆ is hydrogen, C₁₋₄ alkyl, C₁₋₄ haloalkyl, C₁₋₄ alkoxy, C₁₋₄ aminoalkyl or —CO—X wherein X is as defined above.

Preferably R⁶ is hydrogen, C₁₋₄ alkyl, C₁₋₄ haloalkyl, C₁₋₄ alkoxy or C₁₋₄ aminoalkyl. More preferably R⁶ is hydrogen.

Preferred compounds of the invention are those in which:
R¹ is halogen, C₁₋₄ alkyl, C₁₋₂ alkoxy or a moiety -A, -A-A′, -A-Het-A′, -A-L-A′, -A-Het-L-A′, -A-L-Het-A′, -A-Het-L-Het′-A′ or -A-Het-L-Het′-L′;
R² is hydrogen, halogen, C₁₋₄ alkyl, C₁₋₄ alkoxy, C₁₋₄ haloalkyl, C₁₋₄ haloalkoxy or a moiety -Het-L-A′ or -Het-L-Het′-L′;
R³ is hydrogen or C₁₋₂ alkyl;
R⁴ is hydrogen, halogen or C₁₋₄ alkyl;
R⁵ is hydrogen, C₁₋₄ alkyl, C₁₋₄ haloalkyl, C₁₋₄ alkoxy or C₁₋₄ aminoalkyl;
R⁶ is hydrogen, C₁₋₄ alkyl, C₁₋₄ haloalkyl, C₁₋₄ alkoxy, C₁₋₄ aminoalkyl or —CO—X, wherein X is —OR′, —NR′R″ or a 5- to 6-membered heteroaryl or heterocyclyl group which is unsubstituted or substituted with 1 or 2 unsubstituted substituents selected from halogen, C₁₋₄ alkyl, C₁₋₄ haloalkyl or C₁₋₄ alkoxy substitutents, and R′ and R″ are the same or different and represent hydrogen or C₁₋₄ alkyl;
n is zero or 1;
each A is the same or different and is phenyl or a 5- to 6-membered heteroaryl group and is optionally fused to a further phenyl, 5- to 6-membered heteroaryl or 5- to 6-membered heterocyclyl group;
each A′ is the same or different and is a non-fused phenyl, 5- to 6-membered heteroaryl or 5- to 6-membered heterocyclyl;
each Het and Het′ is the same or different and is —O—, —S— or —NR′— where R′ is hydrogen or C₁₋₂ alkyl;
each L is the same or different and is C₁₋₄ alkylene; and
each L′ is the same or different and is hydrogen or a C₁₋₂ alkyl, C₁₋₂ haloalkyl, C₁₋₂ hydroxyalkyl or C₁₋₂ aminoalkyl group;

the phenyl, heteroaryl, heterocyclyl groups in A and A′ being unsubstituted or substituted with 1, 2 or 3 unsubstituted substituents which are the same or different and are selected from halogen, hydroxy, C₁₋₄ alkyl, C₁₋₄ alkoxy, C₁₋₄ haloalkyl and C₁₋₄ haloalkoxy.

Further preferred compounds of the invention are those in which:
R¹ is halogen, C₁₋₄ alkyl, C₁₋₂ alkoxy or a moiety -A, -A-A′, -A-Het-A′, -A-L-A′, -A-Het-L-A′, -A-L-Het-A′, -A-Het-L-Het′-A′ or -A-Het-L-Het′-L′;
R² is hydrogen, halogen, C₁₋₄ alkyl, C₁₋₄ alkoxy, C₁₋₄ haloalkyl, C₁₋₄ haloalkoxy or a moiety -Het-L-A′ or -Het-L-Het′-L′;
R³ is hydrogen or C₁₋₂ alkyl;
R⁴ is hydrogen, halogen or C₁₋₄ alkyl;
R⁵ is hydrogen, C₁₋₄ alkyl, C₁₋₄ haloalkyl, C₁₋₄ alkoxy or C₁₋₄ aminoalkyl;
R⁶ is hydrogen, C₁₋₄ alkyl, C₁₋₄ haloalkyl, C₁₋₄ alkoxy or C₁₋₄ aminoalkyl;
n is zero or 1;
each A is the same or different and is phenyl or a 5- to 6-membered heteroaryl group and is optionally fused to a further phenyl, 5- to 6-membered heteroaryl or 5- to 6-membered heterocyclyl group;
each A′ is the same or different and is a non-fused phenyl, 5- to 6-membered heteroaryl or 5- to 6-membered heterocyclyl;
each Het and Het′ is the same or different and is —O—, —S— or —NR′— where R′ is hydrogen or C₁₋₂ alkyl;
each L is the same or different and is C₁₋₄ alkylene; and
each L′ is the same or different is hydrogen or a C₁₋₂ alkyl, C₁₋₂ haloalkyl or C₁₋₂ hydroxyalkyl group;

the phenyl, heteroaryl, heterocyclyl groups in A and A′ being unsubstituted or substituted with 1, 2 or 3 unsubstituted substituents which are the same or different and are selected from halogen, C₁₋₄ alkyl, C₁₋₄ alkoxy, C₁₋₄ haloalkyl and C₁₋₄ haloalkoxy.

Further preferred compounds of the invention are compounds in which:
R¹ is halogen, C₁₋₄ alkyl, C₁₋₂ alkoxy, -A, -A-A′, -A-O-L-A′ or -A-O-L-Het′-L′;
R² is hydrogen, C₁₋₄ haloalkoxy or a moiety —O-L-A′ or —O-L-Het′-L′;
R³ is hydrogen;
R⁴ is C₁₋₂ alkyl;
n is zero or 1;
R⁵ is hydrogen or C₁₋₄ alkyl;
R⁶ is hydrogen, C₁₋₄ alkyl, C₁₋₄ haloalkyl, C₁₋₄ alkoxy, C₁₋₄ aminoalkyl or —CO—X, wherein X is —OR′, —NR′R″ or a 5- to 6-membered heterocyclyl group which is unsubstituted or substituted by a C₁₋₂ alkyl group and R′ and R″ are the same or different and represent hydrogen or C₁₋₂ alkyl;
each A is the same or different and is a non-fused phenyl or 5- to 6-membered heteroaryl group or is a phenyl ring fused to a further phenyl, 5- to 6-membered heteroaryl or 5- to 6-membered heterocyclyl group, said A group being unsubstituted or substituted with 1 or 2 unsubstituted substituents which are the same or different and are selected from halogen, hydroxy, C₁₋₄ alkyl and C₁₋₄ alkoxy;
each A′ is the same or different and is an unsubstituted 5- to 6-membered heteroaryl or heterocyclyl group;
each Het′ is the same or different and is —O— or —NR′— where R′ is hydrogen or C₁₋₂ alkyl;
each L is the same or different is methylene, n-ethylene or n-propylene; and
each L′ is the same or different and is hydrogen, C₁₋₂ alkyl, C₁₋₂ haloalkyl, C₁₋₂ hydroxyalkyl or C₁₋₂ aminoalkyl.

Typically, in these further preferred compounds of the invention, R₁ is -A, -A-A′, -A-O-L-A′ or -A-O-L-Het′-L″ and each A is a non-fused phenyl group, or is a phenyl group fused to a further phenyl, 5- to 6-membered heteroaryl or 5- to 6-membered heterocyclyl group.

Further preferred compounds of the invention are quinazoline derivatives of formula (Ia) and pharmaceutically acceptable salts thereof:

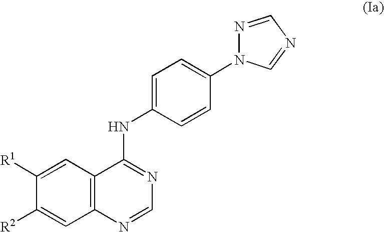

wherein:
R¹ is halogen, C₁₋₄ alkyl, C₁₋₂ alkoxy, -A, -A-A′, -A-O-L-A′ or -A-O-L-Het′-L′;
R² is hydrogen, C₁₋₄ haloalkoxy or a moiety —O-L-A′ or —O-L-Het′-L′;
each A is the same or different and is a phenyl, a 5- to 6-membered heteroaryl or a phenyl fused to a further phenyl, 5- to 6-membered heteroaryl or 5- to 6-membered heterocyclyl group, said A group being unsubstituted or substituted with 1 or 2 unsubstituted substituents which are the same or different and are selected from halogen, C₁₋₄ alkyl and C₁₋₂ alkoxy;
each A′ is the same or different and is an unsubstituted 5- to 6-membered heteroaryl or heterocyclyl group;
each Het′ is the same or different and is —O— or —NR′— where R′ is hydrogen or C₁₋₂ alkyl;
each L is the same or different is methylene, n-ethylene or n-propylene; and
each L′ is the same or different and is hydrogen, C₁₋₂ alkyl or C₁₋₂ hydroxyalkyl.

Further preferred compounds of the invention are quinazoline derivatives of formula (Ib) and pharmaceutically acceptable salts thereof

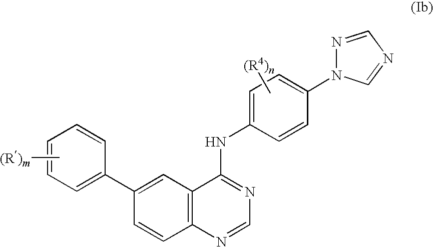

wherein:
each R⁴ is the same or different and represents halogen, C₁₋₄ alkyl or C₁₋₄ alkoxy, preferably C₁₋₄ alkyl, more preferably C₁₋₂ alkyl;
n is 0, 1 or 2;
each R′ is the same or different and represents halogen, C₁₋₄ alkyl or C₁₋₄ alkoxy, preferably C₁₋₂ alkoxy; and
m is 0, 1 or 2.

Further preferred compounds of the invention are quinazoline derivatives of formula (Ic) and pharmaceutically acceptable salts thereof.

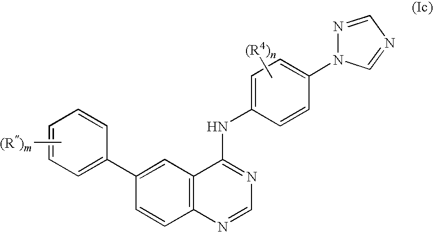

wherein:
each R⁴ is the same or different and represents halogen, C₁₋₄ alkyl or C₁₋₄ alkoxy, preferably C₁₋₄ alkyl, more preferably C₁₋₂ alkyl;
n is 0, 1 or 2; and
R″ is -A′, -Het-A′, -L-A′, -Het-L-A′, -Het-L-Het′-A′ or -Het-L-Het′-L′, wherein A′, Het, L, Het′ and are as defined above.

Particularly preferred compounds of formula (I) include:
1. (6-Iodo-quinazolin-4-yl)-(4-[1,2,4]triazol-1-yl-phenyl)-amine.
2. (6-tert-Butyl-quinazolin-4-yl)-(4-[1,2,4]triazol-1-yl-phenyl)-amine.
3. [7-(3-Chloro-propoxy)-6-methoxy-quinazolin-4-yl]-(4-[1,2,4]triazol-1-yl-phenyl)-amine.
4. [6-Methoxy-7-(3-morpholin-4-yl-propoxy)-quinazolin-4-yl]-(4-[1,2,4]triazol-1-yl-phenyl)-amine.
5. [6-(3,4-Difluoro-phenyl)-quinazolin-4-yl]-(4-[1,2,4]triazol-1-yl-phenyl)-amine.
6. [6-(4-Chloro-3-fluoro-phenyl)-quinazolin-4-yl]-(4-[1,2,4]triazol-1-yl-phenyl)-amine.
7. [6-(3-Fluoro-4-methoxy-phenyl)-quinazolin-4-yl]-(4-[1,2,4]triazol-1-yl-phenyl)-amine.
8. [6-(4-Ethoxy-3-fluoro-phenyl)-quinazolin-4-yl]-(4-[1,2,4]triazol-1-yl-phenyl)-amine
9. {6-[3-Fluoro-4-(2-methoxy-ethoxy)-phenyl]-quinazolin-4-yl}-(4-[1,2,4]triazol-1-yl-phenyl)-amine.
10. [6-(3,4-Dimethoxy-phenyl)-quinazolin-4-yl]-(4-[1,2,4]triazol-1-yl-phenyl)-amine.
11. [6-(3,4-Diethoxy-phenyl)-quinazolin-4-yl]-(4-[1,2,4]triazol-1-yl-phenyl)-amine.
12. {6-[4-(2-Dimethylamino-ethoxy)-phenyl]-quinazolin-4-yl}-(4-[1,2,4]triazol-1-yl-phenyl)-amine.
13. {6-[4-Pyridin-4-ylmethoxy)-phenyl]-quinazolin-4-yl}-(4-[1,2,4]triazol-1-yl-phenyl)-amine.
14. [6-(4-Morpholin-4-yl-phenyl)-quinazolin-4-yl]-(4-[1,2,4]triazol-1-yl-phenyl)-amine.
15. [6-(2,3-Dihydro-benzo[1,4]dioxin-6-yl)-quinazolin-4-yl]-(4-[1,2,4]triazol-1-yl-phenyl)-amine.
16. [6-(2-Methoxy-pyrimidin-5-yl)-quinazolin-4-yl]-(4-[1,2,4]triazol-1-yl-phenyl)-amine.
17. (6-Thiophen-2-yl-quinazolin-4-yl)-(4-[1,2,4]triazol-1-yl-phenyl)-amine.
18. [6-(4-Methyl-thiophen-2-yl)-quinazolin-4-yl]-(4-[1,2,4]triazol-1-yl-phenyl)-amine.
19. (6-Furan-2-yl-quinazolin-4-yl)-(4-[1,2,4-triazol-1-yl-phenyl)-amine.
20. {6-[4-(2-Dimethylamino-ethoxy)-3-fluoro-phenyl]-quinazolin-4-yl}-(4-[1,2,4]triazol-1-yl-phenyl)-amine.
21. (6-Bromo-quinazolin-4-yl)-(4-[1,2,4]triazol-1-yl-phenyl)-amine.
22. N-(2-{2-Fluoro-4-[4-(4-[1,2,4]triazol-1-yl-phenylamino)-quinazolin-6-yl]-phenoxy}-ethyl)-N,N′,N′-trimethyl-ethane-1,2-diamine.
23. [6-(3-Isopropoxy-4-methoxy-phenyl)-quinazolin-4-yl]-(4-[1,2,4]triazol-1-yl-phenyl)-amine.
24. [6-(4-Fluoro-3-isopropoxy-phenyl)-quinazolin-4-yl]-(4-[1,2,4]triazol-1-yl-phenyl)-amine.
25. {6-[3-Fluoro-4-(3-pyrrolidin-1-yl-propoxy)-phenyl]-quinazolin-4-yl}-(4-[1,2,4]triazol-1-yl-phenyl)-amine.
26. {6-[3-Fluoro-4-(3-morpholin-4-yl-propoxy)-phenyl]-quinazolin-4-yl}-(4-[1,2,4]triazol-1-yl-phenyl)-amine.
27. [6-(3,4-Dimethoxy-phenyl)-quinazolin-4-yl]-(2-methyl-4-[1,2,4]triazol-1-yl-phenyl)-amine.
28. 1-(4-{6-[3-Fluoro-4-(2-methoxy-ethoxy)-phenyl]-quinazolin-4-ylamino}-phenyl)-5-methyl-1H-[1,2,4]triazole-3-carboxylic acid.
29. 1-{4-[6-(3,4-Dimethoxy-phenyl)-quinazolin-4-ylamino]-phenyl}-5-methyl-1H-[1,2,4]triazole-3-carboxylic acid.
30. 1-{4-[6-(3,4-Dimethoxy-phenyl)-quinazolin-4-ylamino]-phenyl}-5-methyl-1H-[1,2,4]triazole-3-carboxylic acid dimethylamide.
31. 1-(4-{6-[3-Fluoro-4-(2-methoxy-ethoxy)-phenyl]-quinzoline-4-ylamino}-phenyl)-5-methyl-1H-[1,2,4]triazole-3-carboxylic acid methylamide.
32. 1-(4-{6-[3-Fluoro-4-(2-methoxy-ethoxy)-phenyl]-quinazolin-4-ylamino}-phenyl)-5-methyl-1H-[1,2,4]triazole-3-carboxylic acid dimethylamide.
33. (6-Bromo-quinazolin-4-yl)-(2-methyl-4-[1,2,4]triazol-1-yl-phenyl)-amine.
34. [1-(4-{6-[3-Fluoro-4-(2-methoxy-ethoxy)-phenyl]-quinazolin-4-ylamino}-phenyl)-5-methyl-1H-[1,2,4]triazol-3-yl]-(4-methyl-piperazin-1-yl)-methanone.
35. 2-Methoxy-4-[4-(4-[1,2,4]triazol-1-yl-phenylamino)-quinazolin-6-yl]-phenol.
36. 2-Methoxy-5-[4-(4-[1,2,4]triazol-1-yl-phenylamino)-quinazolin-6-yl]phenol.
37. {6-[3-Fluoro-4-(2-methoxy-ethoxy)-phenyl]-quinazolin-4-yl}-(2-methyl-4-[1,2,4]triazol-1-yl-phenyl)-amine.

and pharmaceutically acceptable salts thereof.

Compounds of formula (I) containing one or more chiral centre may be used in enantiomerically or diastereoisomerically pure form, or in the form of a mixture of isomers. For the avoidance of doubt, the compounds of formula (I) can, if desired, be used in the form of solvates. Further, for the avoidance of doubt, the compounds of the invention may be used in any tautomeric form.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

The compounds of the invention can, for example, be prepared according to the following reaction schemes. In the schemes which follow, the groups R³, R⁵ and R⁶ have, for reasons of clarity, been shown as hydrogen atoms. Similarly, the R⁴ groups have been omitted (i.e. n is zero). Analogous compounds where one or more of R³, R⁵ and R⁶ is other than hydrogen and/or where n is 1 or 2 can be prepared by employing a suitably functionalized 4-triazolylaniline derivative bearing the appropriate substituents in the R³, R⁴, R⁵ and R⁶ positions.

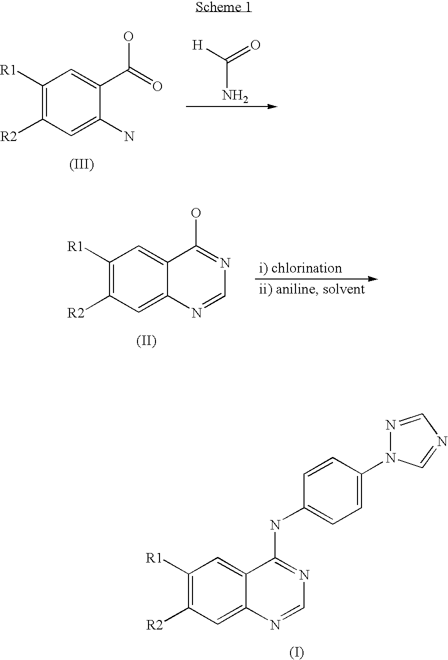

Referring to Scheme 1, the conversion of compounds of formula (II) to compounds of formula (I) is accomplished by converting the 4-hydroxy group of compounds of formula (II) to a suitable leaving group (e.g. chloro) using a reagent such as thionyl chloride as solvent with the addition of a catalytic activator (e.g. dimethylformamide), and subsequent reaction with 4-triazolylaniline in a suitable solvent (e.g. acetonitrile).

Referring to Scheme 1, the conversion of compounds of formula (III) to compounds of formula (II) will be well known to one skilled in the art, being conveniently performed with formamide as solvent and at elevated temperature (e.g. reflux).

Compounds of formula (I) in which R¹ is -A, -A-A′, -A-Het-A′, -A-L-A′, -A-Het-L-A′, -A-L-Het-A′, -A-Het-L-Het′-A′ or -A-Het-L-Het′-L′ can alternatively be produced by the reaction shown in Scheme 2 below. The reaction is typically carried out in the presence of acetic acid at a temperature of about 120° C. and for a period of about 1 hour.

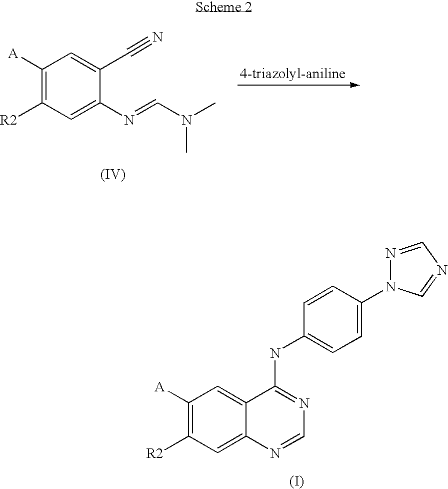

The compounds of formula (IV) used as a starting material in Scheme 2 can be prepared by one of the reactions depicted in Scheme 3 below. In Scheme 3, the groups S1 and S2 may represent protecting groups, such as benzyl, which can be replaced by the desired group by methods known in the art following reaction. Deprotection can be carried out before or after conversion of the compound of formula (IV) to the compound of formula (I).

Referring to Scheme 3, the treatment of compounds of formula (VIII) with an organometallic reagent (VII), or the treatment of compounds of formula (VI) with an organometallic reagent (V), is conveniently carried out in a suitable solvent (such as tetrahydrofuran, dimethylformamide, toluene or propan-2-ol or) and at a suitable temperature (e.g. from ambient to reflux). Conveniently, the reaction is performed under palladium catalysis (e.g. 10 mol % tris (dibenzylideneacetone)dipalladium (II), 10 mol % dichlorobis (triphenylphosphine)palladium (0), 1 mol % bis(benzonitrile)palladium(II) chloride or 0.02 mol % tetrakis(triphenylphosphine)palladium(0)) in the presence of an organic base (e.g. triethylamine) or an inorganic base (e.g. 2N sodium carbonate or potassium phosphate). Where reagent (VII) is an organostannane (e.g. M=SnBu₃), one skilled in the art will recognise the reaction as an example of a Stille coupling where additional additives may be beneficial (e.g. lithium chloride), silver oxide and conveniently the reaction is performed in toluene and at reflux temperature. Where reagent (VII) is a boronic acid derivative, one skilled in the art will recognise the reaction as an example of a Suzuki-Miyaura coupling which may be conveniently performed at 60° C. in propan-2-ol.

As the skilled person in the art will appreciate, the group X in the compounds depicted in Scheme 3 is an appropriate leaving group such as I or Br.

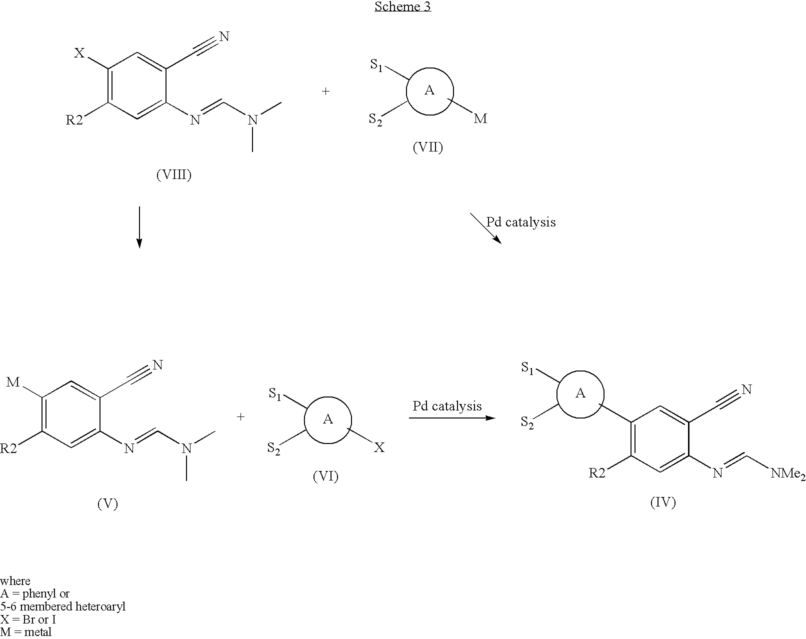

The starting materials in the above reaction scheme are known compounds, or can be prepared by analogy with known methods.

The compounds of the present invention are therapeutically useful. The present invention therefore provides a quinazoline derivative of the formula (I), as defined above, or a pharmaceutically acceptable salt thereof, for use in treating the human or animal body. Also provided is a pharmaceutical composition comprising a quinazoline derivative of the formula (I), as defined above, or a pharmaceutically acceptable salt thereof, and a pharmaceutically acceptable carrier or diluent.

Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

As explained above, the compounds of the invention are active against a flaviviridae infection. The present invention therefore provides the use of a quinazoline derivative of the formula (I), as defined above, or a pharmaceutically acceptable salt thereof, in the manufacture of a medicament for use in treating or preventing a flaviviridae infection. Also provided is a method for treating a patient suffering from or susceptible to a flaviviridae infection, which method comprises administering to said patient an effective amount of a quinazoline derivative of formula (I) or a pharmaceutically acceptable salt thereof.

The flaviviridae family contains three genera. These are hepacivirus, flavivirus and pestivirus. The compounds of the invention are active in treating or preventing a hepacivirus infection, a flavivirus infection or a pestivirus infection.

Typical pestivirus infections which can be treated with the compounds of the invention include bovine viral diarrhea virus, classical swine fever virus and border disease virus.

Typical flavivirus infections which can be treated with the compounds of the invention include yellow fever virus, dengue fever virus, Japanese encephalitis virus and tick borne encephalitis virus.

Typical hepacivirus infections that can be treated with the compounds of the invention include hepatitis C virus.

Compounds of the present invention are especially active against hepatitis C. Typically, said flavivirus is therefore hepatitis C virus.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

Compounds of the present invention may be used in conjunction with known anti-viral agents. Preferred known anti-viral agents in this regard are interferon and ribavirin, and derivatives thereof, which are known for the treatment of hepatitis C (Clinical Microbiology Reviews, January 2000, 67-82). The said medicament therefore typically further comprises interferon or a derivative thereof and/or ribavirin or a derivative thereof. Further, the present invention provides a pharmaceutical composition comprising:
(a) a quinazoline derivative of the formula (I), as defined above, or a pharmaceutically acceptable salt thereof;
(b) interferon or a derivative thereof and/or ribavirin or a derivative thereof; and
(c) a pharmaceutically acceptable carrier or diluent.

Also provided is a product comprising:
(a) a quinazoline derivative of the formula (I), as defined above, or a pharmaceutically acceptable salt thereof; and
(b) interferon or a derivative thereof and/or ribavirin or a derivative thereof,

for separate, simultaneous or sequential use in the treatment of the human or animal body.

A preferred interferon derivative is PEG-interferon. A preferred ribavirin derivative is viramidine.

Further, the compounds of the invention are found to interact synergistically with interferon. Typically, therefore, component (b) of the above pharmaceutical composition or product is interferon, more typically a type I interferon, preferably an interferon α.

A preferred interferon is an interferon α2b, which is typically pegylated, for example PEG-Intron (Schering Plough Corp) or a protein fusion product such as Albuferon (Human Genome Sciences). The interferon α2b may also be formulated for controlled release.

Another preferred interferon is interferon is an interferon α2a. Preferably, the interferon α2a is pegylated, for example Pegasys (Roche) and Roferon A (Roche).

Another preferred interferon is interferon α8 (Riotech).

Another preferred interferon is interferon alfacon-1 (Intermune), preferably pegylated interferon alfacon-1

In another embodiment, the interferon is interferon β, preferably interferon β-1a. (Serono SA).

In another embodiment the interferon is interferon gamma (Intarcia).

Preferably, the interferon is contained in a formulation or device which allows the interferon to be released in a controlled manner. Examples are the DUROS implant technologies developed by ALZA Corp and the SABER drug delivery technology developed by Durect Corp.

The present invention also provides the use of a quinazoline derivative of the formula (I), as defined above, or a pharmaceutically acceptable salt thereof, in the manufacture of a medicament for use in treating or preventing an HCV infection by co-administration with a said interferon or interferon derivative. Also provided is the use of a said interferon or interferon derivative, in the manufacture of a medicament for use in treating or preventing an HCV infection, by co-administration with a quinazoline derivative of the formula (I), as defined above, or a pharmaceutically acceptable salt thereof.

A therapeutically effective amount of a compound of the invention and, when desired, a said interferon or interferon derivative is administered to a patient. A typical dose is from about 0.01 to 100 mg per kg of body weight, according to the activity of the specific compound, the age, weight and conditions of the subject to be treated, the type and severity of the disease and the frequency and route of administration. Preferably, daily dosage levels are from 0.05 to 16 mg per kg of body weight, more preferably, from 0.05 to 1.25 mg per kg of body weight.

The compounds of the present invention may also be used with other antiviral agents. Preferred antiviral agents in this regard are cyclosporins, interleukin 2, interleukin 6, interleukin 12, interfering RNA or antisense RNA, all of which are known for the treatment of Hepatitis C. Compounds of the present invention may also be used in conjunction with other inhibitors of the HCV nucleic acid sequences or HCV protein targets. Preferred HCV protein targets include HCV serine protease as illustrated by VX-950 from Vertex and SCH-503034 from Scering Plough both protease inhibitors, HCV polymerase as illustrated by HCV-796 from Wyeth, HCV helicase, HCV NS4B and HCV NS5A. Further, the compounds of the present invention may also be used in conjunction with inhibitors of HCV entry into the cell or inhibitor of interaction of HCV with host cell proteins.

The following Examples illustrate the invention. They do not however, limit the invention in any way. In this regard, it is important to understand that the particular assay used in the Examples section is designed only to provide an indication of anti-viral activity. There are many assays available to determine such activity, and a negative result in any one particular assay is therefore not determinative.

### EXAMPLES

### Materials and Methods

All temperatures are in ° C. Thin layer chromatography (TLC) was carried out on Si 60G coated plastic plates with uv₂₅₄ indicator (Polygram). All NMR spectra were obtained at 250 MHz in d⁶-DMSO unless stated otherwise, chemical shifts expressed as ppm, apparent coupling constants (J) given for obvious multiplicities.

“Concentrated” implies solvents were removed in vacuo. All solids were dried at 40° C.

### LC-MS Conditions

Samples were run on a MicroMass ZMD, using electrospray with simultaneous positive-negative ion detection.

Column: Synergi Hydro-RP, 30×4.6 mm I.D, 4 μm.

Gradient: 95:5 to 5:95 v/v H₂O/CH₃CN+0.05% Formic Acid over 4.0 min, hold 3 min, return to 95:5 v/v H₂O/CH₃CN+0.05% Formic Acid over 0.2 min and hold at 95:5 v/v H₂O/CH₃CN+0.05% Formic Acid over 3 min.

Detection: PDA 250-340 nm.

Flow rate: 1.5 ml/min.

All retention times (rt) are expressed in minutes.

### Experimental

### Intermediate 1

### 2-amino-5-iodobenzonitrile

Prepared by the method of A. Rosowsky & H. Chen, J. Org. Chem. 2001, 66, 7522-7526.

¹H NMR (CDCl₃) δ 7.64 (1H, s), 7.55 (1H, dd, J 8.5, 2.5 Hz), 6.53 (1H, d, J 8.5 Hz), 4.66 (2H, br s); LC-MS rt 2.42 m/z 243 ES−.

### Intermediate 2

### N′-(2-Cyano-4-iodo-phenyl)-N,N-dimethyl-formamidine

A solution of 2-amino-5-iodobenzonitrile (50 g, 0.2 mol) in DMF-DMA (2.5 eq, 6 ml) was heated to 120° for 2 h. The excess DMF-DMA was removed by concentration to leave the title compound as a viscous brown oil (61 g, quant). Solidifies on standing at 4°.

¹H NMR (CDCl₃) δ 7.79 (1H, d, J 1.9 Hz), 7.65 (1H, dd, J 1.9, 8.5 Hz), 7.57 (1H, s), 6.70 (1H, d, J 8.2 Hz), 3.08 (6H, s); LC-MS rt 2.1 M/z 300 ES+

### Intermediate 3

### 2-amino-5-bromobenzonitrile

A solution of 2-aminobenzonitrile (11.8 g, 0.1 mol) in AcOH (120 ml) was treated with ammonium bromide (10.3 g, 0.105 mol) and hydrogen peroxide (10.2 ml, 35% in water, 0.105 mol). This mixture was stirred at room temperature for ca. 24 hours until LCMS analysis showed the reaction was complete. The mixture was concentrated to remove the AcOH, prior to stirring the residue with 30% aqueous NaOH solution until basic. The resulting solid was removed by filtration and washed with water before drying. This solid was then dissolved in an excess of DCM. The solution was concentrated until precipitation started and then allowed to stand until crystallisation was complete. The resulting solid was removed by filtration and washed with a little DCM. This gave the desired compound as an off white crystalline solid (19.2 g, 97%).

¹H NMR δ 7.61 (1H, d, J 2.5 Hz), 7.43 (1H, dd, J 9, 2.5 Hz), 6.75 (1H, d, J 9 Hz), 6.28 (2H, br s); LC-MS rt 2.24

### Intermediate 4

### N′-(4-bromo-2-cyano-phenyl)-N,N-dimethyl-formamidine

A solution of 2-amino-5-bromobenzonitrile (10.25 g, 52 mmol) in DMF-DMA (20 ml) was heated to reflux for 1 hour. The mixture was cooled and concentrated to dryness before triturating with t-butyl-methyl-ether (TBME) (10 ml). Petrol (30 ml) was then added and the solid scratched, and allowed to stand for 1 hour. The solid product was filtered off and washed with TBME/petrol (1:2) to afford a crystalline solid (11.95 g, 91.2%).

¹H NMR (CDCl₃) δ 7.55 (1H, d, J 2 Hz), 7.51 (1H, s), 7.41 (1H, dd, J 2, 9 Hz), 6.75 (1H, d, J 9 Hz), 3.01 (6H, s); LC-MS rt 1.95 m/z 252/254 ES−.

### Intermediate 5

### 2-Amino-5-(4,4,5,5-tetramethyl-[1,3,2]dioxaborolan-2-yl)-benzonitrile

A mixture of Pd Cl₂ (dppf) (3.35 g), potassium acetate (12.07 g) and bis(pinacolato)boron (12.48 g) in dry DMF (80 ml) was treated with Intermediate 1 (10 g) and heated to 80° for 4 h. The cooled mixture was partitioned between water (400 ml) and DCM (400 ml). The aqueous phase was further extracted with DCM (2×100 ml) and the combined organic phases dried and concentrated. The residue was purified by chromatography on silica gel (90 g, MPLC) with 10-30% EtOAc in petrol as eluant. Concentration of fractions containing product and trituration with further petrol gave the desired product as a white solid (6.91 g, 69%)

¹H NMR (CDCl₃) δ 7.87 (1H, s), 7.72 (2H, d, J 8.21), 6.7 (1H, d, J 8.2 Hz), 4.57 (2H, br s), 1.31 (12H, s); LC-MS rt 2.84 rt 244 ES+.

### Intermediate 6

### N′-[2-Cyano-4-(4,4,5,5-tetramethylty-[1,3,2]dioxaborolan-2-yl)-phenyl]-N,N-dimethyl-formamidine

A suspension of Intermediate 5 (750 mg) in DMF-DMA (1 ml) was heated to 100° under N₂ for 30 mins then cooled to rt. The solvent was removed and the residue purified by SPE on silica gel (5 g) with 10% EtOAc/petrol as eluant. This gave the title compound as a clear oil which crystallised on standing (915 mg, 100%).

¹H NMR (CDCl₃) 7.98 (1H, s), 7.817 (1H, d, J 8.2 Hz), 7.62 (1H, s), 6.92 (1H, d, J 7.6 Hz), 3.1 (3H, s), 3.07 (3H, s), 1.33 (12H, s); LC-MS rt 2.73 m/z 300 ES+

### Intermediate 7

### 4-(4-Iodo-phenoxymethyl)-pyridine

A mixture of 4-iodophenol (1 g) K₂CO₃ (powdered, 1.88 g) and 4-picolyl chloride (822 mg) in acetone (15 ml) was heated at reflux for 16 h. A further portion of 4-picolyl chloride (411 mg) added and heating continued for 6 h. Cooled, filtered and the filtrate adsorbed onto silica and purified by MPLC (35 g Si, 10-50% EtOAc in petrol gradient elution over 30 min). This gave, on concentration, a white solid (710 mg, 50%).

¹H NMR (CDCl₃) δ 8.64 (2H, d, J 5.69 Hz), 7.6 (2H, d, J 8.85 Hz), 7.35 (2H, d, J 5.69 Hz), 6.76 (2H, d, J 8.85 Hz), 5.08 (2H, s).

### Intermediate 8

### 4-Bromo-1,2-diethoxy-benzene

To a well stirred mixture of diethoxybenzene (500 mg) and ammonium bromide (323 mg, 1.1 eq) in MeCN (20 ml) was added oxone (2.03 g, 1.1 eq). This suspension was stirred at rt for 4 h then the suspension filtered and the filtrate concentrated to give the title compound (723 mg,>90%) which was used without further purification.

¹H NMR (CDCl₃) 6.98 (2H, m), 6.73 (1H, d, J 8.85 Hz), 4.05 (4H, m), 1.44 (6H, m); LC-MS rt 2.48 m/z 279 ES+.

### Intermediate 9

### 4-Bromo-1-(2-bromoethoxy)-2-fluorobenzene

To a stirred solution of 4-bromo-2-fluorophenol (3.5 g, 18.3 mmol, 1 eq), 2-bromoethanol (3.44 g, 27.5 mmol, 1.5 eq) and triphenylphosphine (7.21 g, 27.5 mmol, 1.5 eq) in THF (50 mL) at 0° C., under nitrogen, was added DEAD (4.78 g, 27.5 mmol, 1.5 eq) drop-wise via syringe. The reaction was then allowed to warm to room temperature. After 2 h the reaction was concentrated to dryness in vacuo and the off-white residue placed on top of a short pad of silica and washed with 9:1 petroleum spirit:EtOAc (3×50 mL). The filtrate was concentrated to give the title compound as a clear oil (5.28 g, 97%).

¹H-NMR (DMSO-d₆). 3.57 (t, 2H), 4.26 (t, 2H), 6.80 (m, 1H), 7.12 (m, 7.19 (m, 1H). LC-MS rt 2.90 m/z no ion.

### Intermediate 10

### N-[2-(4-bromo-2-fluorophenoxy)ethyl]-N′,N′,N′-trimethylethane-1,2-diamine

A mixture of Intermediate 9 (3 g, 10 mmol, 1 eq), N,N,N′-trimethylethylenediamine (1.54 g, 15 mmol, 1.5 eq) and potassium carbonate (2.09 g, 15 mmol, 1.5 eq) in acetone was heated at reflux. After 16 h the reaction was allowed to cool to room temperature and then filtered. The filtrate was concentrated in vacuo to give a pale orange syrup. The syrup was purified by flash column chromatography, eluting initially with 100% CH₂Cl₂ and then 100:8:1 CH₂Cl₂:EtOH:NH₃. The tile compound was isolated as a pale orange syrup (2.24 g, 70%). R_{f}=0.12 (100:8:1 CH₂Cl₂:EtOH:NH₃).

¹H-NMR (DMSO-d₆) 2.41 (s, 3H), 2.57 (s, 6H), 2.82 (m, 4H), 2.91 (t, 2H), 4.14 (t, 2H), 6.89 (m, 1H), 7.21 (m, 1H), 7.27 (m, 1H). LC-MS rt 1.79 m/z 320 ES+.

### Intermediate 11

### N′-(3-cyano-4′-{2-[(2-dimethylaminoethyl)methylamino]ethoxy}-3′-fluorobiphenyl-4-yl)—N,N-dimethylformamidine

A mixture of Intermediate 10 (950 mg, 2.98 mmol, 1 eq), bis(pinacolato)diboron (1.51 g, 5.95 mmol, 2 eq), potassium acetate (1.02 g, 10.43 mmol, 3.5 eq) and PdCl₂(dppf)₂CH₂Cl₂ (245 mg, 0.30 mol, 0.1 eq) in DMF (10 mL) was heated at 80° C., under nitrogen. After 16 h the reaction was allowed to cool to room temperature and concentrated in vacuo to give a brown residue. The residue was extracted with EtOAc (3×20 mL) and the extracts were combined and concentrated in vacuo to dryness to give a brown oil (1.09 g). A mixture of the oil (1.09 g), N′-(2-cyano-4-iodophenyl)-N,N-dimethylformamide (811 mg, 2.71 mmol) and tetrakis(triphenylphosphine)palladium (162 mg, 0.14 mmol) in DME (10 mL) and a saturated aqueous solution of Na₂CO₃ (5 mL) was heated at reflux. After 20 h the reaction was allowed to cool to room temperature and then concentrated in vacuo to give a brown residue. The residue was purified by flash column chromatography eluting with 800:8:1, 200:8:1 and 100:8:1 CH₂Cl₂:EtOH:NH₃. The title compound was isolated as a pale brown oil (273 mg, 22%). R_{f}=0.08 (100:8:1 CH₂Cl₂:EtOH:NH₃).

¹H-NMR (DMSO-d₆) 2.20 (s, 6H), 2.33 (s, 3H), 2.41 (t, 2H), 2.57 (t, 2H), 2.81 (t, 2H), 3.03 (s, 3H), 3.05 (s, 3H), 4.11 (t, 2H), 6.95 (m, 2H), 7.17 (m, 2H), 7.58 (m, 2H), 7.61 (m, 1H). LC-MS rt 1.86 m/z 412 ES+.

### Intermediate 12

### 2-Methoxy-5-bromophenol

Prepared by the method of Meyers and Snyder, J. Org. Chem., 1993, 58, 1, 42.

¹H NMR (CDCl₃) 7.00 (1H, d, J 2.25 Hz), 6.90 (1H, dd, J 8.5, 2.25 Hz), 6.66 (1H, d, J 8.75 Hz), 5.57 (1H, s), 3.81 (3H, s)

### Intermediate 13

### 5-Bromo-2-fluorophenol

Prepared by the method of M Elliott, N Janes & B Khambay, GB2187731.

¹H NMR (CDCl₃) 7.08 (1H, m), 6.89 (2H, m), 5.14 (1H, s)

### Intermediate 14

### 4-Bromo-2-isopropoxy-1-methoxy-benzene

To a solution of Intermediate 12 (279 mg, 1.37 mmol) in DMF (5 ml) was added potassium carbonate (945 mg, 6.85 mmol) and 2-iodopropane (684 μl, 6.85 mmol). The reaction mixture was stirred at 90° C. over 4 hrs. Analysis by LC-MS showed 20% starting phenol remaining so another portion of 2-iodopropane (684 μl, 6.85 mmol) was added and the mixture stirred at 90° C. overnight.

LC-MS analysis showed consumption of all starting phenol so the reaction mixture was filtered and the filtrate was diluted with water (50 ml) and extracted into ethyl acetate three times. The combined extracts were dried over magnesium sulphate, filtered and evaporated under reduced pressure yielding a dark brown oil (294 mg, 87%).

¹H NMR (D⁶-DMSO) 6.93 (2H, m), 6.68 (1H, d, J 8.75 Hz), 4.43 (1H, m), 3.75 (3H, s), 1.31 (6H, d, J 6 Hz); LC-MS rt 2.80.

### Intermediate 15

### 4-Bromo-1-fluoro-2-isopropoxy-benzene

To a solution of Intermediate 13 (262 mg, 1.37 mmol) in DMF (5 ml) was added potassium carbonate (945 mg, 6.85 mmol) and 2-iodopropane (684 μl, 6.85 mmol). The reaction mixture was stirred at 90° C. over 4 hrs. Analysis by LCMS showed 20% starting phenol remaining so another portion of 2-iodopropane (684 μl, 6.85 mmol) was added and the mixture stirred at 90° C. overnight.

LCMS analysis showed consumption of all starting phenol so the reaction mixture was filtered and the filtrate was diluted with water (50 ml) and extracted into ethyl acetate three times. The combined extracts were dried over magnesium sulphate, filtered and evaporated under reduced pressure yielding a dark brown oil (274 mg, 86%).

¹H NMR (D⁶-DMSO) 7.00 (1H, dd, J 2.25 Hz), 6.92 (2H, m), 4.45 (1H, m), 1.30 (6H, d, J 6.25 Hz)

LC-MS rt 2.93

### Intermediate 16

### 4-Amino-3′-isopropoxy-4′-methoxy-biphenyl-3-carbonitrile

To a solution of Intermediate 14 (294 mg, 1.2 mmol) in dimethoxyethane (4 ml) was added Intermediate 5 (439 mg, 1.8 mmol) followed by sat. aq. Sodium carbonate (2 ml) and tetrakis(triphenylphosphine)palladium (0) (139 mg, 0.12 mmol). This reaction mixture was stirred at 80° C. overnight. LCMS analysis showed consumption of starting material so reaction mixture was diluted with water and extracted into dichloromethane three times. The combined extracts were dried over magnesium sulphate, filtered and evaporated under reduced pressure. Column chromatography (gradient 0-50% EtOAc in petroleum ether) furnished the desired product as a yellow solid (137 mg, 40%).

¹H NMR (CDCl₃) 7.47 (2H, m), 6.94 (2H, m), 6.87 (1H, d, J 9 Hz), 6.75 (1H, d, J 8.5 Hz), 4.55 (1H, quin, J 6.25 Hz), 4.37 (2H, s), 3.81 (3H, s), 1.34 (6H, d, J 6.25 Hz);

LC-MS rt 2.70

### Intermediate 17

### 4-Amino-4′-fluoro-3′-isopropoxy-biphenyl-3-carbonitrile

To a solution of Intermediate 15 (274 mg, 1.18 mmol) in dimethoxyethane (4 ml) was added Intermediate 5 (439 mg, 1.8 mmol) followed by sat. aq. Sodium carbonate (2 ml) and tetrakis(triphenylphosphine)palladium (0) (139 mg, 0.12 mmol). This reaction mixture was stirred at 80° C. overnight.

LCMS analysis showed consumption of starting material so reaction mixture was diluted with water and extracted into dichloromethane three times. The combined extracts were dried over magnesium sulphate, filtered and evaporated under reduced pressure. Column chromatography (gradient 0-50% EtOAc in petroleum ether) furnished the desired product as a yellow solid (196 mg, 60%).

¹H NMR (CDCl₃) 7.41 (2H, m), 6.97 (3H, m), 6.72 (2H, d, J 8 Hz), 4.53 (1H, quin, J 6 Hz), 4.40 (2H, s), 1.33 (6H, d, J 6 Hz); LC-MS rt 2.86

### Intermediate 18

### N′-(3-Cyano-3′-isopropoxy-4′-methoxy-biphenyl-4-yl)-N,N-dimethyl-formamidine

A solution of Intermediate 16 (137 mg, 0.41 mmol) in DMF-DMA (2 ml) was stirred at 80° C. overnight. LCMS analysis shows consumption of starting material. Reaction mixture evaporated under reduced pressure and then redissolved in toluene and evaporated under reduced pressure again. Column chromatography (gradient 20-40% EtOAc in petroleum ether) furnished the desired compound as a dark yellow oil which solidified on standing (120 mg, 87%)

¹H NMR (CDCl₃) 7.63 (1H, d, J 2 Hz), 7.57 (1H, s), 7.54 (1H, dd, J 8.5 Hz, 2.25 Hz), 7.00 (2H, s), 6.91 (2H, m), 4.54 (1H, quin, J 6 Hz), 3.82 (1H, s), 3.05 (6H, d, J=6.5 Hz), 1.35 (6H, d, J 6 Hz); LC-MS rt 2.40 m/z 338 ES+

### Intermediate 19

### N′-(3-Cyano-4′-fluoro-3′-isopropoxy-biphenyl-4-yl)-N,N-dimethyl-formamidine

A solution of Intermediate 17 (196 mg, 0.60 mmol) in DMF-DMA (2 ml) was stirred at 80° C. overnight. LCMS analysis shows consumption of starting material Reaction mixture evaporated under reduced pressure and then redissolved in toluene and evaporated under reduced pressure again. Column chromatography (gradient 20-40% EtOAc in petroleum ether) furnished the desired compound as a dark yellow oil which solidified on standing (190 mg, 97%).

¹H NMR (CDCl₃) 7.57 (2H, m), 7.49 (1H, dd, J 8.5 Hz, 2.25 Hz), 6.99 (4H, m), 4.53 (1H, quin, J 6 Hz), 3.02 (6H, d, J 5.75 Hz), 1.32 (6H, d, J 6 Hz); LC-MS rt 2.71 m/z 326 ES+

### Intermediate 20

### 1-(3-Methyl-4-nitro-phenyl)-1H-[1,2,4]triazine

To a 50 ml round bottom flask was added the 4-fluoro-2-methyl-1-nitrobenzene (0.5 g, 3.22 mmol), Na₂CO₃ (0.36 g, 3.38 mmol) and 1,2,4-triazole (0.22 g, 3.22 mmol) in DMF (DRY 10 ml). The mixture was stirred at 125° C. for 24 hr under nitrogen. The DMF was evaporated to dryness and the crude product was put on a silica column and eluted with 2.5% MeOH:DCM. Isolated 0.47 g (72%) of a white solid.

¹H n.m.r (D₆-DMSO) 9.52 (1H, s), 8.39 (1H, s), 8.27 (1H, d, J=8.85 Hz), 8.13 (1H, d, J=1.89 Hz), 8.02 (1H, dd, J=8.85 Hz, 2.53 Hz), 2.68 (3H, s); LC-MS rt 2.26 m/z 205 ES+

### Intermediate 21

### 2-Methyl-4-[1,2,4]triazol-1-yl-phenylamine

To a 50 ml round bottom flask was added Intermediate 20 (0.47 g, 2.3 mmol) and EtOH (10 ml). To this stirred mixture at room temperature was added SnCl₂.2H₂O (2.5 g, 11.5 mmol). The mixture was then heated at 80° C. for 4 hr. The mixture was allowed to cool and the pH of the solution was taken to 8 via addition of 2N NaOH. The mixture was filtered and concentrated to dryness then partitioned between DCM:H₂O (50 ml:25 ml). The aqueous layer was separated and washed again with DCM (25 ml). The DCM fractions were combined and put through a hydrophobic frit to remove water. The filtrate was concentrated to dryness to afford a brown solid 0.3 g (75%).

¹H n.m.r (D₆-DMSO) 9.02 (1H, s), 8.15 (1H, s), 7.43 (1H, d, J=1.89 Hz), 7.37 (1H, dd, J=8.20 Hz, 2.52 Hz), 6.76 (1H, d, J=8.85 Hz), 5.20 (2H, s), 2.18 (3H, s); LC-MS rt 1.33 m/z 175 ES+

### Intermediate 22

### N′-[3-Cyano-3′-fluoro-4′-(2-methoxy-ethoxy)-biphenyl-4-yl]-N,N-dimethyl-formamidine

### Step 1: 4-Bromo-2-fluoro-1-(2-methoxy-ethoxy)-benzene

A mixture of 4-fluorophenol (9.8 ml), powdered potassium carbonate (2 eq, 24 g) and bromoethyl methyl ether (1.1 eq, 20.16 ml) in acetone (60 ml) was heated to reflux overnight. The cooled reaction mixture was diluted with water and extracted into EtOAc. The combined organic phases were washed with aqueous sodium carbonate, dried (Na₂SO₄) and concentrated to give the product as a mobile oil (22 g, quantitative).

¹H n.m.mr. (CDCl₃) δ 7.14 (2H, m), 6.84 (1H, t, J 8.85 Hz), 4.09 (2H, m), 3.69 (2H, m), 3.38 (3H, s); LC-MS rt 2.67 m/z no ion

### Step 2: 3-Fluoro-4-(2-methoxy-ethoxy)-phenylboronic acid

To a solution of 4-bromo-2-fluoro-1-(2-methoxy-ethoxy)-benzene (4.98 g) in THF (40 ml) was added a small crystal of iodine and then Mg (730 mg, 1.5 eq) portion-wise. After addition, the mixture was heated to reflux for 4 h. The grey mixture was cooled to −78° trimethylborate (1.2 eq, 2.25 ml) added and allowed to warm overnight. 1N HCl (aq, 60 ml) was added and stirred for 30 min before being extracted into ether (2×50 ml). The combined organic phases were dried and concentrated and the resulting solid triturated with ether/petrol, isolated by filtration and dried to give the title compound as a cream solid (3.122 g, 73%)

¹H NMR (CDCl₃) δ 8.04 (2H, br s), 7.53 (2H, m), 7.12 (1H, t, J 8.85 Hz), 4.17 (2H, m), 3.67 (2H, m), 3.3 (3H, s); LC-MS rt 1.97 m/z 213 ES−

### Step 3: N′-[3-Cyano-3′-fluoro-4′-(2-methoxy-ethoxy)-biphenyl-4-yl]-N,N-dimethyl-formamidine

A mixture of 3-fluoro-4-(2-methoxyethoxy)-phenylboronic acid (1.83 g, 1.2 eq), intermediate 2 (2.32 g), potassium carbonate (1.29 g, 1.2 eq) in DMF/H₂O (3:1, 40 ml) was treated with dichloro(bis benzonitrile) palladium (II) (1%, 30 mg) and stirred at ambient under N₂ for 4 h. The mixture was diluted with water (100 ml) and filtered then extracted with EtOAc (2×50 ml) and these organic extracts added to the filter cake, dried and concentrated. The residue was dissolved in DCM, loaded onto a short column of silica and eluted portion-wise under suction with DCM/EtOH/NH₃ 400-200:8:1. On concentration, this gave a brown oil which was triturated with DCM/ether/petrol and on crystallization diluted with further petrol (60 ml). The title compound was isolated by filtration as a cream solid (1.665 g, 63%)

¹H NMR (CDCl₃) δ 7.74 (1H, d, J 1.9 Hz), 7.7 (1H, s), 7.60 (1H, dd, J 8.85, 1.9 Hz), 7.29 (3H, m), 7.1 (1H, d, J 8.2 Hz), 7.05 (1H, d, J 8.85 Hz), 4.28 (2h, m), 3.84 (2H, m), 3.52 (3H, s), 3.16 (3H, s), 3.14 (3H, s); LC-MS rt 2.32 m/z 342 ES+

### Intermediate 23

### N′-(3-Cyano-3′,4′-dimethoxy-biphenyl-4-yl)-N,N-dimethyl-formamidine

Intermediate 4 (2.52 g, 10 mmol) and 3,4-dimethoxyphenyl boronic acid (1.9 g, 1.2 eq) in iPrOH (30 ml) were treated with 2N aqueous sodium carbonate (10 ml) and tetrakis-(triphenylphosphine) palladium (0) (5 mg, 0.04 mol %) and heated with stirring at 60°. After 2 hours the reaction was shown to be ca. 80% complete by LCMS. The reaction mixture was then cooled and diluted with water (30 ml). The resulting solid was filtered off, washed with further water (2×20 ml) before drying by suction. The solid was re-slurried twice in ether (2×5 ml) sucked dry, and finally dried to give the biphenyl intermediate as an off white solid (2.24 g, 72%).

¹H NMR (D⁶-DMSO) δ 7.99 (1H, s) 7.91 (1H, d, J 1.9 Hz), 7.79 (1H, dd, J 8.2, 1.9 Hz), 7.2 (3H, m), 6.99 (1H, d, J 8.2 Hz), 3.84 (3H, s), 3.78 (3H, s), 3.08 (3H, s) 3.01 (3H, s); LC-MS rt 2.31 m/z 309 ES−

### Intermediate 24

### 1-(4-Amino-phenyl)-5-methyl-1H-[1,2,4]triazole-3-carboxylic acid

A solution of 5-methyl-1-(4-nitrophenyl)-1H-1,2,3-triazole-3-carboxylic acid (100 mg, 0.403 mM) in methanol (8 ml) was injected at a 1 ml/min rate into an hydrogenator “the H-Cube” that combines endogenous hydrogen generation with a disposable cartridge system (Pd/C), temperature was set up to 25° C. at atmospheric pressure.

The solution obtained was concentrated to give a white solid (82 mg, 93%).

¹H NMR (D⁶-DMSO) δ 8.72 (1H, broad s), 8.59 (1H, broad s), 7.42 (2H, d, J 8 Hz), 7.23 (1H, d, J 8 Hz), 6.99 (2H, d, J 8 Hz), 6.70 (1H, d, J 8 Hz), 2.48 (3H, s), 2.45 (3H, s).

### Intermediate 25

### 1-(4-Amino-phenyl)-5-methyl-1H-[1,2,4]triazole-3-carboxylic acid dimethylamide

Step 1: 5-Methyl-1-(4-nitrophenyl)-1H-[1,2,4]triazole-3-carboxylic acid dimethylamide.

To a solution of 5-methyl-1(4-nitrophenyl)-1H-1,2-4-triazole-3-carboxylic acid (Key organics 400 mg, 1.61 mM) and Hunig's base (667 ul, 3.86 mM) in DCM:DMF (6 ml:1 ml) at −10° C. was added dropwise isobutylchloroformate (250 ul, 1.93 mM). The reaction was stirred 30 min. at −10° C. then slowly a 2M solution of dimethylamine (965 ul, 1.93 mM) was added. The mixture was allowed to warm-up to room temperature and stirred for another hour. The crude mixture was washed with sat. NaHCO₃ extracted and dried over MgSO₄. After evaporation the yellow solid obtained was used directly in the next reaction without further purification (412 mg, 93%). LC-MS rt 2.09 m/z 275 ES+

¹H NMR (D⁶-DMSO) δ 8.42 (2H, d, J 7 Hz), 7.95 (2H, d, J 7 Hz), 3.13 (3H, s), 3.03 (3H, s), 2.62 (3H, s).

### Step 2: 1-(4-Amino-phenyl-5-methyl-1H-[1,2,4]triazole-3-carboxylic acid dimethylamide

A solution of 5-methyl-1-(4-nitrophenyl)-1H-1,2,3-triazole-3-carboxylic acid methylamide from step 1 (100 mg, 0.36 mM) in methanol (8 ml) was injected at a 1 ml/min rate into an hydrogenator “the H-Cube” that combines endogenous hydrogen generation with a disposable cartridge system (Pd/C), temperature was set up to 25° C. at atmospheric pressure.

The solution obtained was concentrated to give a white solid (87 mg, 98%). LC-MS: rt 1.54 m/z 245 ES⁺

¹H NMR (D⁶-DMSO) δ 8.71 (1H, broad s), 8.54 (2H, d, J 7 Hz), 7.12 (2H, d, J 7 Hz), 3.13 (3H, s) 3.03 (3H, s), 2.62 (3H, s).

### Example 1

### (6-Iodo-quinazolin-4-yl)-(4-[1,2,4]triazol-1-yl-phenyl)-amine

4-Chloro-6-iodoquinazoline (WO9609294 A1, 150 mg) was treated with 4-triazolyl aniline (28 mg, 1 eq) in MeCN and refluxed for 6 h. Cooled overnight and filtered to give the title compound (70 mg).

¹H NMR δ 11.75 (1H, br s), 9.4 (2H, s), 9.04 (1H, s), 8.43 (111, d, J 8.85 Hz), 8.34 (1H, s), 8.0 (4H, m), 7.82 (1H, d, J 8.85 Hz); LC-MS rt 2.32 m/z 415 ES+.

### Example 2

### (6-tert-Butyl-quinazolin-4-yl)-(4-[1,2,4]triazol-1-yl-phenyl)-amine

A solution of 2-amino-5-tert-butylbenzoic acid (commercial sources, 500 mg) and formamidine acetate (404 mg) in EtOH (5 ml) was refluxed for 18 h. The cooled mixture was filtered and the precipitate washed with ice-cold EtOH and dried to give the hydroxy quinazoline (394 mg) which was added to thionyl chloride (10 ml) and DMF (cat.) and heated to reflux overnight. The cooled mixture was diluted with EtOAc and poured onto sat sodium bicarbonate (aq). The organic phase was separated, dried and concentrated to a brown solid (327 mg), of which a portion (105 mg) was treated immediately with 4-triazolylaniline (125 mg) in MeCN (4 ml) at reflux overnight. The cooled mixture was partitioned between DCM and sodium bicarbonate and the organic phase concentrated. Purification by chromatography with DCM:EtOH:NH₃ (200:8:1) as eluant gave the desired compound.

¹H NMR δ 10.16 (1H, s), 9.48 (1H, s), 8.77 (1H, s), 8.62 (1H, s), 8.41 (1H, d), 8.2 (4H, m), 7.95 (1H, d), 1.64 (9H, s); LC-MS rt 2.18 m/z 343 ES−.

### Example 3

### [7-(3-Chloro-propoxy)-6-methoxy-quinazolin-4-yl]-(4-[1,2,4]triazol-1-yl -phenyl)amine

N′-[5-(3-Chloro-propoxy)-2-cyano-4-methyoxy-phenyl]-N,N-dimethyl-formamidine (WO03055491, 580 mgs), 4-(1,2,4-triazolyl)aniline (314 mgs) and AcOH (4 mL) were heated to 90° for 1 hr before cooling. The oil was concentrated and the resultant slurry dissolved in MeOH, sonicated and the resultant white powder filtered and dried to produce the title compound (765 mg).

¹H NMR δ 9.65 (s, 1H), 9.25 (s, 1H), 8.5 (s, 1H), 8.23 (s, 1H), 8.02 (d, 2H, J 9 Hz), 7.89 (s, 1H), 7.87 (d, 2H, J=9), 7.24 (s, 1H), 4.28 (t, 2H, J=6), 3.95 (s, 3H), 3.83 (t, 2H, J 6 Hz), 2.27 (q, 211, J 6 Hz); LC-MS rt 2.13 m/z 411 ES+.

### Example 4

### [6-Methoxy-7-(3-morpholin-4-yl-propoxy)-quinazolin-4-yl]-(4-[1,2,4]triazol-1-yl-phenyl)-amine

Example 3 (118.6 mgs, 0.289 mmol), morpholine (120 μL) and dimethylacetamide (1.1 mL) were heated to 90° for 12 hr before cooling, concentrating and purifying the resultant oil by column chromatography with 200:8:1,

DCM:MeOH:NH₃ as eluant. The oil was recrystallised from MeCN producing the title compound as colourless crystals, (126 mg).

¹H NMR δ 9.55 (s, 1H), 8.57 (s, 1H), 8.02 (s, 1H), 7.80 (d, 2H, J 8.9) 7.35 (s, 1H), 7.19 (d, 2H, J=8.9), 4.38 (t, 2H, J 6 Hz), 4.15 (s, 3H), 4-3.92 (m, 4H), 3.83-3.75 (m, 4H), 3.36-3.28 (m, 41-1); LC-MS rt 1.78 m/z 462 ES+

### Example 5

### [6-(3,4-Difluoro-phenyl)-quinazolin-4-yl]-(4-[1,2,4]triazol-1-yl-phenyl)-amine

### Step 1: N′-(3-Cyano-3′,4′-difluoro-biphenyl-4-yl)-N,N-dimethyl-formamidine

A mixture of 3,4-difluorophenyl boronic acid (Lancaster, 396 mg, 1.5 eq), Intermediate 2 (500 mg) and tetrakis(triphenylphosphine)palladium (0) (5%, 96 mg) was heated in DME/2N sodium carbonate (aq, 2:1, 13.5 ml) at reflux for 16 h. The mixture was concentrated, the residue washed with water and ether and dried to give a light brown solid (386 mg, 81%).

¹H NMR (CDCl₃) δ 7.57 (1H, d, J=2 Hz), 7.54 (1H, s), 7.45 (111, dd, J=8.5, 2.25 Hz), 7.13 (3H, m), 6.90 (1H, d, J=8.5 Hz), 3.0 (3H, s), 2.99 (3H, s); LC-MS rt 2.51; m/z 286 ES+.

### Step 2: [6-(3,4-Difluoro-phenyl)-quinazolin-4-yl]-(4-[1,2,4]triazol-1-yl-phenyl)-amine

A mixture of formamidine from Step 1 (150 mg) and 4-triazolyl-aniline (88 mg, 1 eq) in AcOH (2 ml) was heated to 80° for 16 h. Cooled, concentrated and cautiously treated with sodium bicarbonate (aq). The resulting solid was isolated by filtration, washed with water then DCM:EtOH:NH₃ (20:8:1) added and refiltered. The filtrate was concentrated until a precipitate formed which was filtered, washed with ether and dried to give the title compound (82 mg, 34%).

¹H NMR δ 10.0 (1H, s), 9.18 (1H, s), 8.75 (1H, s), 8.54 (1H, s), 8.12 (2H, m), 7.97 (1H, m), 7.92 (2H, m), 7.78 (3H, m), 7.66 (1H, m), 7.53 (1H, m); LC-MS rt 2.52 m/z 401 ES+.

### Example 6

### [6-(4-Chloro-3-fluoro-phenyl)-quinazolin-4-yl]-(4-[1,2,4]triazol-1-yl-phenyl)-amine

### Step 1: N′-(4′-Chloro-3-cyano-3′-fluoro-biphenyl-4-yl)-N,N-dimethyl-formamidine

A mixture of 4-chloro-3-fluorophenyl boronic acid (Combiblocks, 759 mg, 1.3 eq), Intermediate 2 (1 g) and tetrakis(triphenylphosphine) palladium (0) (5%, 193 mg) was heated in DME/2N sodium carbonate (aq, 2:1, 27 ml) at reflux for 16 h. The mixture was filtered, the filtrate concentrated, the residue washed with sat sodium bicarbonate, water and ether and dried to give the product (310 mg)

¹H NMR (CDCl₃) δ 7.58 (1H, d, J 2.0 Hz), 7.53 (1H, s), 7.46 (1H, dd, J 8.5, 2.25 Hz), 7.31 (1H, t, J 8.0 Hz), 7.14 (1H, m), 7.12 (1H, m), 6.9 (1H, d, J 8.5 Hz), 3.0 (3H, s), 2.98 (3H, s); LC-MS rt 2.70; m/z 302 ES+.

### Step 2: [6-(4-Chloro-3-fluoro-phenyl)-quinazolin-4-yl]-(4-[1,2,4]triazol-1-yl-phenyl)-amine

A mixture of formamidine from Step 1 (300 mg) and 4-triazolyl-aniline (167 mg, 1 eq) in AcOH (3 ml) was heated to 80° for 2 h. Cooled, and the resulting solid was isolated by filtration, washed with sodium bicarbonate, water and MeCN. The solid was purified by column chromatography on silica gel with DCM:EtOH:NH₃ (400:8:1 to 200:8:1) as eluant to give the title compound.

¹H NMR δ 10.36 (1H, br s), 9.29 (1H, s), 8.93 (1H, s), 8.63 (1H, s), 8.25 (2H, m), 8.03 (3H, m), 7.86 (2H, d, J 9.25 Hz), 7.81 (3H, m); LC-MS rt 2.61 m/z 418 ES+.

### Example 7

### [6-(3-Fluoro-4-methoxy-phenyl)-quinazolin-4-yl]-(4-[1,2,4]triazol-1-yl-phenyl)-amine

### Step 1: N′-(3-Cyano-3′-fluoro-4′-methoxy-biphenyl-4-yl)-N,N-dimethyl-formamidine

A mixture of 3-fluoro-4-methoxyphenyl boronic acid (Aldrich, 427 mg, 1.5 eq), Intermediate 2 (500 mg) and tetrakis(triphenylphosphine)palladium (0) (5%, 96 mg) was heated in DME/2N sodium carbonate (aq, 2:1, 13.5 ml) at reflux for 16 h. The mixtures were concentrated, the residue washed with water and ether and dried to give a light brown solid (436 mg, 88%).

¹H NMR (CDCl₃) δ 7.71 (1H, d, J=2.25 Hz), 7.67 (1H, s), 7.59 (1H, dd, J=9.12 2.25 Hz), 7.32 (1H, m), 7.27 (1H, m), 7.05 (2H, m), 3.96 (3H, s), 3.14 (3H, s), 3.12 (3H, s); LC-MS rt 2.33 m/z 298 ES+.

### Step 2: [6-(3-Fluoro-4-methoxy-phenyl)-quinazolin-4-yl]-(4-[1,2,4]triazol-1-yl-phenyl)-amine

A mixture of formamidine from Step 1 (200 mg) and 4-triazolyl-aniline (112 mg, 1 eq) in AcOH (2 ml) was heated to 80° for 16 h. Cooled, concentrated and cautiously treated with sodium bicarbonate (aq). The resulting solid was isolated by filtration, washed with water then DCM:EtOH:NH₃ (20:8:1) added and refiltered. The filtrate was concentrated until a precipitate formed which was filtered, washed with ether and dried to give the title compound (129 mg, 47%).

¹H NMR δ 10.06 (1H, s), 9.29 (1H, s), 8.81 (1H, s), 8.63 (1H, s), 8.23 (2H, m), 8.07 (2H, d, J 10.0 Hz), 7.93 (1H, s), 7.88 (2H, m), 7.83 (1H, m), 7.73 (1H, d, J 7.50 Hz), 7.35 (1H, t, J 8.75 Hz), 3.92 (3H, s); LC-MS rt 2.42 m/z 413 ES+.

### Example 8

### [6-(4-Ethoxy-3-fluoro-phenyl)-quinazolin-4-yl]-(4-[1,2,4]triazol-1-yl-phenyl)-amine

### Step 1: N′-(3-Cyano-4′-ethoxy-3′-fluoro-biphenyl-4-yl)-N,N-dimethyl-formamidine

A mixture of 4-ethoxy-3-fluorophenyl boronic acid (Combiblocks, 800 mg, 1.3 eq), Intermediate 2 (1 g) and tetrakis(triphenylphosphine) palladium (0) (5%, 193 mg) was heated in DME/2N sodium carbonate (aq, 2:1, 27 ml) at reflux for 16 h. The mixture was filtered, the filtrate concentrated, the residue washed with sat sodium bicarbonate, water and ether and dried to give the product (410 mg).

¹H NMR δ 7.71 (1H, d, J=2.0 Hz), 7.67 (1H, s), 7.60 (1H, dd, J=8.5 2.25 Hz), 7.31 (1H, m), 7.25 (1H, m), 7.02 (2H, m), 4.17 (2H, q, 7.0 Hz), 3.14 (3H, s), 3.12 (3H, s), 1.5 (3H, t, 7.0 Hz); LC-MS rt 2.51 m/z 312 ES+.

### Step 2: [6-(4-Ethoxy-3-fluoro-phenyl)-quinazolin-4-yl]-(4-[1,2,4]triazol-1-yl-phenyl)-amine

A mixture of formamidine from Step 1 (400 mg) and 4-triazolyl-aniline (214 mg, 1 eq) in AcOH (3 ml) was heated to 80° for 2 h. Cooled, and the resulting solid was isolated by filtration, washed with sodium bicarbonate, water and MeCN and dried to give pure title compound.

¹H NMR δ 10.27 (1H, br s), 9.29 (1H, s), 8.86 (1H, s), 8.63 (1H, s), 8.26 (1H, s), 8.22 (1H, dd, J 8.75 2.5 Hz), 8.10 (2H, d, J 7.50 Hz), 7.88 (4H, m), 7.72 (1H, d, 7.5 Hz), 7.34 (1H, t, 7.5 Hz), 4.21 (2H, q, 7.5 Hz), 1.41 (3H, t, 7.5 Hz); LC-MS rt 2.53 m/z 427 ES+.

### Example 9

### {6-[3-Fluoro-4-(2-methoxy-ethoxy)-phenyl]-quinazolin-4-yl}-(4-[1,2,4]triazol-1-yl-phenyl)-amine

### Step 1: 4-Bromo-2-fluoro-1-(2-methoxy-ethoxy)-benzene

A mixture of 4-fluorophenol (9.8 ml), powdered potassium carbonate (2 eq, 24 g) and bromoethyl methyl ether (1.1 eq, 20.16 ml) in acetone (60 ml) heated to reflux overnight. The cooled reaction mixture was diluted with water and extracted into EtOAc. The combined organic phases were washed with aqueous sodium carbonate, dried (Na₂SO₄) and concentrated to give the product as a mobile oil (22 g, quantitative).

¹H NMR (CDCl₃) δ 7.14 (2H, m), 6.84 (1H, t, J 8.85 Hz), 4.09 (2H, m), 3.69 (2H, m), 3.38 (3H, s); LC-MS rt 2.67 m/z no ion.

### Step 2: 3-Fluoro-4-(2-methoxy-ethoxy)-phenylboronic acid

To a solution of 4-bromo-2-fluoro-1-(2-methoxy-ethoxy)-benzene (4.98 g) in THF (40 ml) was added a small crystal of iodine and then Mg (730 mg, 1.5 eq) portion-wise. After addition, the mixture was heated to reflux for 4 h. The grey mixture was cooled to −78° trimethylborate (1.2 eq, 2.25 ml) added and allowed to warm overnight. 1N HCl (aq, 60 ml) was added and stirred for 30 min before being extracted into ether (2×50 ml). The combined organic phases were dried and concentrated and the resulting solid triturated with ether/petrol, isolated by filtration and dried to give the title compound as a cream solid (3.122 g, 73%).

¹H NMR δ 8.04 (2H, br s), 7.53 (2H, m), 7.12 (1H, t, J 8.85 Hz), 4.17 (2H, m), 3.67 (2H, m), 3.3 (3H, s); LC-MS rt 1.97 m/z 213 ES−.

### Step 3: N′-[3-Cyano-3′-fluoro-4′-(2-methoxy-ethoxy)-biphenyl-4-yl]-1N,N-dimethyl-formamidine

A mixture of 3-fluoro-4-(2-methoxyethoxy)-phenylboronic acid (1.83 g, 1.2 eq), intermediate 2 (2.32 g), potassium carbonate (1.29 g, 1.2 eq) in DMF/H₂O (3:1, 40 ml) was treated with dichloro(bis benzonitrile) palladium (II) (1%, 30 mg) and stirred at ambient under N₂ for 4 h. The mixture was diluted with water (100 ml) and filtered then extracted with EtOAc (2×50 ml) and these organic extracts added to the filter cake, dried and concentrated. The residue was dissolved in DCM, loaded onto a short column of silica and eluted portion-wise under suction with DCM/EtOH/NH₃ 400-200:8:1. On concentration, this gave a brown oil which was triturated with DCM/ether/petrol and on crystallization diluted with further petrol (60 ml). The title compound was isolated by filtration as a cream solid (1.665 g, 63%).

¹H NMR (CDCl₃) δ 7.74 (1H, d, J 1.9 Hz), 7.7 (1H, s), 7.60 (1H, dd, J 8.85, 1.9 Hz), 7.29 (3H, m), 7.1 (1H, d, J 8.2 Hz), 7.05 (1H, d, J 8.85 Hz), 4.28 (2h, m), 3.84 (2H, m), 3.52 (3H, s), 3.16 (3H, s), 3.14 (3H, s); LC-MS rt 2.32 m/z 342 ES+.

### Step 4: {6-[3-Fluoro-4-(2-methoxy-ethoxy)-phenyl]-quinazolin-4-yl}-(4-[1,2,4]triazol-1-yl-phenyl)-amine

A mixture of formamidine from Step 3 (594 mg) and 4-triazolyl-aniline (279 mg, 1 eq) in AcOH (6 ml) was heated to 125° for 2 h. On cooling and dilution with water (20 ml), a yellow precipitate was isolated by filtration, slurried with 1N NaOH and washed with water. After drying, this material was triturated with MeOH/water/acetone˜10:5:5 to give, after filtration and drying, a pale cream solid (510 mg, 64%).

¹H NMR δ 10.1 (1H, br s), 9.27 (1H, s), 8.8 (1H, s), 8.58 (1H, s), 8.24 (1H, s), 8.18 (1H, d, J 8.85 Hz), 8.03 (2H, d, J 8.85 Hz), 7.84 (4H, m), 7.68 (1H, d, J 9.5 Hz), 7.35 (1H, t, J 8.85 Hz), 4.26 (2H, m), 3.72 (2H, m), 3.35 (3H, obscured by H₂O); LC-MS rt 2.4 m/z 457 ES+.

### Example 10

### [6-(3,4-Dimethoxy-phenyl)-quinazolin-4-yl]-(4-[1,2,4]triazol-1-yl-phenyl)-amine

### Step 1: N′-(3-Cyano-3′,4′-dimethoxy-biphenyl-4-yl)-N,N-dimethyl-formamidine

Intermediate 4 (2.52 g, 10 mmol) and 3,4-dimethoxyphenyl boronic acid (1.9 g, 1.2 eq) in iPrOH (30 ml) were treated with 2N aqueous sodium carbonate (10 ml) and tetrakis-(triphenylphosphine) palladium (0) (5 mg, 0.04 mol %) and heated with stirring at 60°. After 2 hours the reaction was shown to be ca. 80% complete by LCMS. The reaction mixture was then cooled and diluted with water (30 ml). The resulting solid was filtered off, washed with further water (2×20 ml) before drying by suction. The solid was re-slurried twice in ether (2×5 ml) sucked dry, and finally dried to give the biphenyl intermediate as an off white solid (2.24 g, 72%).

¹H NMR δ 7.99 (1H, s) 7.91 (1H, d, J 1.9 Hz), 7.79 (1H, dd, J 8.2, 1.9 Hz), 7.2 (3H, m), 6.99 (1H, d, J 8.2 Hz), 3.84 (3H, s), 3.78 (3H, s), 3.08 (3H, s) 3.01 (3H, s); LC-MS rt 2.31 m/z 309.

### Step 2: [6-(3,4-Dimethoxy-phenyl)-quinazolin-4-yl]-(4-[1,2,4]triazol-1-yl-phenyl)-amine

Formamidine from step 1(2.96 g, 9.6 mmol) and 4-triazolylaniline (1.54 g, 9.6 mmol) in AcOH (10 ml) were heated to reflux for 3 h. The cooled solution was diluted with ether (200 ml) and the resulting precipitate filtered off. The filter cake was washed with ether, dried under suction then slurried with 2N NaOH (100 ml) with stirring for 30 minutes. The resulting solid was again isolated by filtration, washed with water and dried to give a pale yellow solid (3.81 g, 93%).

¹H NMR δ 10.11 (1H, s), 9.30 (1H, s), 8.80 (1H, s), 8.65 (1H, s), 8.26 (1H, s), 8.23 (1H, s), 8.10 (2H, d, J 10 Hz), 7.92 (2H, d, J 10 Hz), 7.88 (1H, d, J 7.5 Hz), 7.47 (2H, overlapping s), 7.16 (1H, d, J 7.5 Hz), 3.93 (3H, s), 3.85 (3H, s); LC-MS rt 2.17 m/z 425 ES+.

### Example 11

### [6-(3,4-Diethoxy-phenyl)-quinazolin-4-yl]-(4-[1,2,4]triazol-1-yl-phenyl)-amine

A mixture of Intermediate 8 (200 mg) and Intermediate 6 (490 mg) were combined with terakis(triphenylphosphine) palladium (0) (95 mg) in DME (3 ml) and sodium carbonate (1 ml) and heated to 100° overnight. The cooled mixture was diluted with water and extracted into DCM. The organic phases were combined, concentrated and partially purified by column chromatography with CH₂Cl₂/EtOH/NH₃ (200:8:1) to give coupled material. A portion of this material (70 mg) was heated in acetic acid (1 ml) with 4-triazolylaniline (37 mg) at 80° over 1 h. The mixture was concentrated, basified with sat. NaHCO₃ and the resulting precipitate isolated by filtration and washed with water and ether, dried then washed with EtOAc, MeCN, then ether and dried to give the title compound (32 mg, 34%).

¹H NMR δ 10.4 (1H, br s), 9.27 (1H, s), 8.82 (1H, s), 8.6 (1H, s), 8.23 (1H, s), 8.16 (2H, d, J 8.85 Hz), 7.95 (2H, d, J 8.85 Hz), 7.44 (2H, m), 7.13 (1H, d, J 8.2 Hz), 4.15 (4H, m), 1.37 (6H, m); LC-MS rt 2.44, m/z 453 ES+.

### Example 12

### {6-[4-(2-Dimethylamino-ethoxy)-phenyl]-quinazolin-4-yl}-(4-[1,2,4]triazol-1-yl-phenyl)-amine

### Step 1: N′-[3-Cyano-4′-(2-dimethylamino-ethoxy)-biphenyl-4-yl]-N,N-dimethyl-formamidine

4-(2-dimethylamino-ethoxy)-boronic acid (prepared as per C. Zhou and R. C. Larock, Journal of Organic Chemistry, Vol. 70, No. 10, pp 3765, 915 mg, 1.2 eq), intermediate 2 (934 mg), and potassium carbonate (1.2 eq, 520 mg) in DMF/H₂O (3:1, ml) was treated with dichloro(bis benzonitrile) palladium (II) (1%, 12 mg) and stirred at ambient under N₂ for 4 h. The mixture was concentrated and partitioned between water and EtOAc. The combined organic phases were dried and concentrated before being loaded onto an SPE (20 g, Si) and eluted portionwise under suction with a DCM—DCM/EtOH/NH₃—200:8:1 gradient. On concentration, this gave a brown oil (1.0 g, quant).

¹H NMR (CDCl₃) δ 7.71 (1H, d, J 2.5 Hz), 7.64 (1H, s), 7.6 (1H, dd, J 8.2, 2.5 Hz), 7.44 (2H, d, 8.2 Hz), 6.98 (3H, m), 4.1 (2H, m), 3.11 (3H, s), 3.08 (3h, s) 2.75 (2H, m), 2.29 (6H, s); LC-MS rt 1.71 m/z 337 ES+.

### Step 2: {6-[4-(2-Dimethylamino-ethoxy)-phenyl]-quinazolin-4-yl}-(4-[1,2,4]triazol-1-yl phenyl)-amine

A mixture of the formamidine from step 1 (528 mg) and 4-triazolylaniline (294 mg) in AcOH (5 ml) was heated to 125° for 3 h. After cooling, the mixture was diluted and basified with 1N NaOH (80 ml). The resulting cream ppt was isolated by filtration, and dried to give the title compound (495 mg, 70%).

¹H NMR δ 10.3 (1H, br s), 9.34 (1H, s), 8.9 (1H, s), 8.67 (1H, s), 8.31 (1H, s), 8.23 (1H, dd, J 8.85, 1.9 Hz), 8.15 (2H, d, J 8.85 Hz), 7.92 (4H, m), 7.19 (2H, d, J 8.85 Hz), 4.2 (2H, m), 2.72 (2H, m), 2.3 (6H, s); LC-MS rt 1.96 m/z 452 ES+.

### Example 13

### {6-[4-Pyridin-4-ylmethoxy)-phenyl]-quinazolin-4-yl}-(4-[1,2,4]triazol-1-yl-phenyl)-amine

### Step 1: 4-Amino-4′-(pyridin-4-ylmethoxy)-biphenyl-3-carbonitrile

A mixture of Intermediate 5 (835 mg, 1.5 eq), Intermediate 7 (710 mg) and tetrakis(triphenylphosphine)palladium (0) (5%, 263 mg) was heated in DME/2N sodium carbonate (aq, 2:1, 15 ml) at 80° for 16 h. Aqueous workup between water and EtOAc gave a residue that was purified MPLC (35 g Si, 10-100% EtOAc in petrol gradient elution over 30 min). This gave, on concentration, the title compound (310 mg, 45%).

¹H NMR (CDCl₃) δ 8.63 (2H, m), 7.5 (2H, m), 7.38 (3H, m), 6.99 (2H, d, J 8.65 Hz), 6.79 (1H, d, J 8.65 Hz), 5.127 (2H, s), 4.42 (2H, br s).

### Step 2: {6-[4-Pyridin-4-ylmethoxy)-phenyl]-quinazolin-4-yl}-(4-[1,2,4]triazol-1-yl-phenyl)-amine

Step 1 intermediate (310 mg) was treated with DMF-DMA (5 ml) in DMF (3 ml) at 80° for 3 h. The mixture was evaporated, dissolved in toluene and concentrated to a pale yellow solid which was dried overnight. A portion of this material (74 mg) was treated with 4-triazolylaniline (57 mg) in AcOH (1 ml) at 80° for 2 h. The cooled mixture was basified with aq sodium bicarbonate and the resulting precipitate filtered, washed with water, ether and MeCN to give the title compound (88 mg, 59%).

¹H NMR δ 10.5 (1H, brs), 9.26 (1H, s), 8.87 (1H, s), 8.59 (3H, m), 8.23 (1H, s), 8.1 (3H, m), 7.84 (5H, m) 7.47 (5H, m), 7.18 (2H, m), 5.29 (2H, s); LC-MS rt 2.23 m/z 472 ES+.

### Example 14

### [6-(4-Morpholin-4-yl-phenyl)-quinazolin-4-yl]-(4-[1,2,4]triazol-1-yl-phenyl-amine

### Step 1: N′-(3-Cyano-4′-morpholin-4-yl-biphenyl-4-yl)-N,N-dimethyl-formamidine

A mixture of 4-morpholinylphenyl boronic acid (Maybridge, 992 mg, 1.5 eq), Intermediate 2 (955 mg) and tetrakis(triphenylphosphine)palladium (0) (5%, 185 mg) was heated in DME/2N sodium carbonate (aq, 2:1, 12 ml) at 90° for 16 h. Aqueous workup between water and EtOAc gave a brown residue that was purified by SPE (Si, 20 g) with portionwise elution under suction with DCM/EtOH/NH₃ 600-200:8:1. This gave a brown solid that was triturated with DCM/petrol and filtered to give a light brown solid (430 mg, 40%).

¹H NMR (CDCl₃) δ 7.45 (3H, m), 7.29 (2H, d, J 8.85 Hz), 6.81 (3H, m), 3.71 (4H, m), 3.03 (4H, m), 2.94 (3H, s), 2.91 (3H, s); LC-MS rt 2.16 m/z 335 ES+.

### Step 2: [6-(4-Morpholin-4-yl-phenyl)-quinazolin-4-yl]-(4-[1,2,4]triazol-1-yl-phenyl)-amine

A mixture of the formamidine from step 1 (76 mg) and 4-triazolylaniline (24 mg) in AcOH (2 ml) was heated to 125° for 1 h. After cooling, the mixture was diluted and basified with 1N NaOH (20 ml). The resulting ppt was isolated by filtration, washed with DCM then triturated with MeOH/acetone. The filtrate was concentrated to give the title compound (22 mg) as a light green solid.

¹H NMR δ 10.45 (1H, br s), 9.3 (1H, s), 8.88 (1H, s), 8.6 (1H, s), 8.26 (1H, s), 8.14 (3H, m), 7.87 (5H, m), 7.12 (2H, d, J 8.85 Hz), 3.78 (4H, m), 3.21 (4H, m); LC-MS rt 2.28 m/z 450 ES+.

### Example 15

### [6-(2,3-Dihydro-benzo[1,4]dioxin-6-yl)-quinazolin-4-yl]-(4-[1,2,4]triazol-1-yl-phenyl)-amine

### Step 1: N′-[2-Cyano-4-(2,3-dihydro-benzo[1,4]dioxin-6-yl)-phenyl]-N,N-dimethyl-formamidine

1,4-(Ethylenedioxy)benzene-6-boronic acid (Lancaster, 785 mg, 1.2 eq), Intermediate 2 (1.08 g) and tetrakis(triphenylphosphine)palladium (0) (205 mg) was heated in DME/2N sodium carbonate (aq, 2:1, 12 ml) at 80° for 18 h. Aqueous workup between water and EtOAc gave a brown solid that was purified by SPE (Si, 20 g) with portionwise elution under suction with DCM/EtOH/NH₃ 600-200:8:1. This gave a light brown solid (430 mg, 41%).

¹H NMR (CDCl₃) δ 7.68 (1H, d, J 2.5 Hz), 7.63 (1H, s), 7.56 (1H, dd, J 8.2, 2.5 Hz), 6.96 (4H, m), 4.29 (4H, s), 3.1 (3H, s), 3.08 93H, s). LC-MS rt 2.22 m/z 308 ES+.

### Step 2: [6-(2,3-Dihydro-benzo[1,4]dioxin-6-yl)-quinazolin-4-yl]-(4-[1,2,4]-triazol-1-yl-phenyl)-amine

A mixture of the formamidine from step 1 (304 mg) and 4-triazolylaniline (75 mg) in AcOH (2 ml) was heated to 125° for 1.5 h. After cooling, the mixture was diluted and basified with 1N NaOH (20 ml). The resulting ppt was isolated by filtration and dried to give the title compound (136 mg, 71%) as a yellow solid.

¹H NMR δ 10.07 (1H, s), 9.28 (1H, s), 8.78 (1H, s), 8.62 (1H, s), 8.25 (1H, s), 8.16 (1H, d, J 8.85 Hz), 8.07 (2H, d, J 8.85 Hz), 7.86 (2H, d, J 8.85 Hz), 7.82 (1H, d, J 8.85 Hz), 7.47 (1H, d, J 1.9 Hz), 7.38 (1H, dd, J 8.2, 1.9 Hz), 7.03 (1H, d, J 8.85 Hz) 4.32 (4H, s); LC-MS rt 2.35 m/z 423 ES+.

### Example 16

### [6-(2-Methoxy-pyrimidin-5-yl)-quinazolin-4-yl]-(4-[1,2,4]triazol-1-yl-phenyl)-amine

### Step 1: 2-Amino-5-(2-methoxy-pyrimidin-5-yl)-benzonitrile

A mixture of Intermediate 3 (602 mg), 2-methoxypyrimidine-5-boronic acid (Frontier, 1.5 eq, 706 mg), and tetralds(triphenylphosphine)palladium (0) (352 mg) was heated in DME/2N sodium carbonate (aq, 2:1, 20 ml) at 100° for 12 h. Aqueous workup between water and EtOAc gave a brown solid that purified by SPE (Si, 20 g) by elution with DCM (4×10 ml) then EtOAc (4×10 ml), to give a solid that was triturated with DCM/petrol, and filtered to give a light brown solid (660 mg).

¹H NMR δ 8.85 (2H, s), 7.8 (1H, s), 7.6 (>3H, m), 6.88 (1H, d, J 8.85 Hz), 6.3 (2H, s), 3.93 (3H, s); LC-MS rt 2.39 m/z 226 ES−.

### Step 2: [6-(2-Methoxy-pyrimidin-5-yl)-quinazolin-4-yl]-(4-[1,2,4]triazol-1-yl-phenyl)-amine

The product from step 1 (645 mg) was heated in DMF-DMA (4 ml) for 2 h. On cooling the mixture was concentrated and triturated with ether/petrol to give the formamidine as a solid (664 mg). A portion of this material (56 mg) was treated with 4-triazolyl-aniline (35 mg) in AcOH (1.5 ml) at 100° for 2 h, cooled and diluted with water (25 ml). Basified with NaOH, filtered and the solid dried to give the title compound (66 mg).

¹H NMR δ 10.04 (1H, s), 9.24 (1H, s), 9.13 (2H, s), 8.86 (1H, s), 8.63 (1H, s), 8.25 (2H, m), 8.04 (2H, m), 7.89 (3H, m), 3.99 (3H, s); LC-MS rt 2.32 m/z 395 ES−.

### Example 17

### (6-Thiophen-2-yl-quinazolin-4-yl)-(4-[1,2,4]triazol-1-yl-phenyl)-amine

### Step 1: N′-(2-Cyano-4-thiophen-2-yl-phenyl)-N,N-dimethyl-formamidine

A mixture of Intermediate 2 (2 g), lithium chloride (1.42 g), dichlorobis-(triphenylphosphine) palladium (II) (0.235 g) and 2-(tributylstannyl)thiophene (2.74 g) in toluene (40 ml) was heated to 120° for 24 h. The cooled reaction mixture was concentrated and loaded onto a column of silica gel and eluted with DCM:MeOH (2.5%) to give the title compound (0.4 g).

¹H NMR δ 8.02 (1H, s), 7.88 (1H, d, J 1.9 Hz), 7.74 (1H, dd, J 8.85, 2.5 Hz), 7.52 (1H, s), 7.50 (1H, s), 7.2 (1H, d, J 8.85 Hz), 7.11 (1H, t, J 4.4 Hz), 3.09 (3H, s), 3.0 s); LC-MS rt 2.32 m/z 256 ES+.

### Step 2: (6-Thiophen-2-yl-quinazolin-4-yl)-(4-[1,2,4]triazol-1-yl-phenyl)-amine

A mixture of formamidine from Step 1 (151 mg) and 4-triazolylaniline (100 mg) in AcOH (5 ml) were heated to 125° for 3 h. The cooled reaction mixture was basified with 2N NaOH and the precipitate isolated by filtration and purified by column chromatography on silica gel with DCM:MeOH (2.5%) as eluant. This gave the title compound (200 mg).

¹H NMR δ 10.1 (1H, s), 9.3 (1H, s), 8.83 (1H, s), 8.63 (1H, s), 8.26 (1H, s), 8.19 (1H, d, J 8.85 Hz), 8.07 (2H, d, J 8.85 Hz), 7.92 (2H, d, J 8.85 Hz), 7.85 (1H, d, J 8.85 Hz), 7.76 (1H, d, J 2.5 Hz), 7.70 (1H, d, J 5 Hz), 7.27 (1H, m); LC-MS rt 2.39 m/z 369 ES−.

### Example 18

### [6-(4-Methyl-thiophen-2-yl)-quinazolin-4-yl]-(4-[1,2,4]triazol-1-yl-phenyl)-amine

### Step 1: 2-Amino-5-(4-methyl-thiophen-2-yl)-benzonitrile

A mixture of Intermediate 3 (1 g), 4-methyl-2-thienylboronic acid (Acros, 2 eq, 1.44 g), and tetrakis(triphenylphosphine)palladium (0) (586 mg) was heated in DME/2N sodium carbonate (aq, 2:1, 30 ml) at 100° for 2.5 h. Aqueous workup between water and EtOAc gave a brown solid that was triturated with DCM/petrol, filtered and washed with ether to give a light brown solid (865 mg, 80%).

¹H NMR δ 7.6 (1H, d, J 2.52 Hz), 7.54 (1h, dd, J 8.85, 2.5 Hz), 7.16 (1H, s), 6.98 (1H, s), 6.80 (1H, d, J 8.85 Hz), 6.25 (2H, br s), 2.19 (3H, s); LC-MS rt 2.91 m/z 215 ES+.

### Step 2: N′-[2-Cyano-4-(4-methyl-thiophen-2-yl)-phenyl]-N,N-dimethyl-formamidine

The product from step 1 (850 mg) was heated in DMF-DMA (1.32 ml) for 1.5 h. On cooling the mixture was diluted with ether, filtered and washed with further ether to give the formamidine as a grey solid (564 mg). A portion of this material (54 mg) was treated with 4-triazolyl-aniline (35 mg) in AcOH (1.5 ml) at 100° for 2 h, cooled and diluted with water (25 ml). Basified with NaOH, filtered and the solid dried to give the title compound (54.4 mg, 70%).

¹H NMR δ 10.0 (1H, s), 9.18 (1H, s) 8.67 (1H, s), 8.51 (1H, s), 8.14 (1H, s), 8.0 (3H, m), 7.8 (2H, d), 7.72 (1H, d, J 8.85 Hz), 7.48 (1H, s), 7.15 (1H, s), 2.2 (3H, s); LC-MS rt 2.72 m/z 385 ES+.

### Example 19

### (6-Furan-2-yl-quinazolin-4-yl)-(4-[1,2,4-triazol-1-yl-phenyl)-amine

### Step 1: N′-(2-Cyano-4-furan-2-yl-phenyl)-N,N-dimethyl-formamidine

A mixture of Intermediate 2 (1 g), lithium chloride (0.71 g), dichlorobis(triphenylphosphine) palladium (II) (0.717 g) and 2-(tributylstannyl)furan (1.31 g) in toluene (25 ml) was heated to 90° for 24 h. The cooled reaction mixture was concentrated and loaded onto a column of silica gel and eluted with DCM, followed by DCM:MeOH (2.5%) to give the product (0.32 g).

¹H NMR δ 8.05 (1H, s), 7.91 (1H, d, J 1.9 Hz), 7.8 (1H, dd, J 8.85, 1.9 Hz), 7.73 (1H, d, J 1.9 Hz), 7.25 (1H, d, J 8.85 Hz), 6.95 (1H, d, J 3.2 Hz), 6.58 (1H, m), 3.1 (3H, s), 3.02 (3H, s); LC-MS rt 2.04 m/z 240 ES−.

### Step 2: (6-Furan-2-yl-quinazolin-4-yl)-(4-[1,2,4-triazol-1-yl-phenyl)-amine

The formamidine from Step 1 (100 mg) and 4-triazolylaniline (73 mg) in AcOH (3 ml) were heated at 125° for 3 h. The cooled reaction mixture was concentrated and purified by column chromatography on silica gel with DCM:MeOH (2.5%) as eluant. This gave the product (54 mg).

¹H NMR δ 10.14 (1H, s), 9.28 (1H, s), 8.87 (1H, s), 8.62 (1H, s), 8.25 (2H, m), 8.06 (2H, m), 7.9 (4H, m), 7.16 (1H, m), 6.73 (1H, m); LC-MS rt 2.25 m/z 353 ES−.

### Example 20

### {6-[4-(2-Dimethylamino-ethoxy)-3-fluoro-phenyl]-quinazoline-4-yl}-(4-[1,2,4]triazole-1-yl-phenyl)amine

### Step 1: [2-(4-Bromo-2-fluoro-phenoxy)-ethyl]dimethyl-amine

A solution of 4-Bromo-2-Fluorophenol (20 mmol, 2.19 ml) dimethylaminoethylchloride.HCl (25 mmol, 3.6 g), powdered potassium carbonate (80 mmol, 11.06 g) in dry ethanol (200 ml) and dry toluene (200 ml) heated to 80° overnight. The cooled reaction mixture was concentrated under vacuum and partitioned between ethyl acetate and water. The combined organic phases were washed with aqueous sodium carbonate, dried (Na₂SO₄) and concentrated to give the product as a yellow oil. Purification by chromatography with DCM:EtOH:NH3 (200:8:1) as eluant gave the desired compound as a pale oil (4.11 g, 78%).

### Step 2: 3-Fluoro-4-(2-dimethylamine-ethoxy)-phenylboronic acid

To a solution of [2-(4-Bromo-2-fluoro-phenoxy)-ethyl]-dimethyl-amine from step 1 (4.1 g) in THF (40 ml) was added a small crystal of iodine and then Mg (570 mg, 1.5 eq) portion-wise. After addition, the mixture was heated to reflux for 1 h. The grey mixture was cooled to −78° trimethylborate (1.2 eq, 2.1 ml) added drop-wise and allowed to warm overnight. 1N HCl (aq, 60 ml) was added and stirred for 30 min before being extracted into ether (2×50 ml). The combined organic phases were dried and concentrated and the resulting solid triturated with acetone/ether, isolated by filtration and dried to give the title compound as a brown solid (2.6 g, 73%). The product was used crude without further purification.

### Step 3: N′-[3-Cyano-3′-fluoro-4′-(2-methoxy-ethoxy)-biphenyl-4-yl]-N,N-dimethyl-formamidine

A mixture of 3-fluoro-4-(2-dimethylamine-ethoxy)-phenylboronic acid (1.96 g, 1.2 eq), intermediate 2 (2.14 g), potassium carbonate (1.19 g, 1.2 eq) in DMF/H₂O (3:1, 20 ml) was treated with dichloro(bis benzonitrile) palladium (II) (1%, 27 mg) and stirred at ambient under N₂ for 2 h. The mixture was diluted with water (200 ml) and filtered then extracted with ethyl acetate (2×50 ml) and these organic extracts added to the filter cake, dried and concentrated to a brown solid.
The residue was dissolved in DCM, loaded onto a short column of silica and eluted portion-wise under suction with DCM/EtOH/NH₃ 400-200:8:1. On concentration, this gave a beige solid (1.88 g, 74%).

LC-MS rt 1.75 m/z 355 ES+.

### Step 4: {6-[4-(2-Dimethylamino-ethoxy)-3-fluoro-phenyl]-quinazoline-4-yl}-(4-[1,2,4]triazole-1-yl-phenyl)amine

To a solution of formamidine from Step 3 (250 mg, 0.73 mM) in acetic acid (2 ml) was added the 4-triazolylaniline (117 mg, 0.73 mM). The reaction was heated to 125° C. for 2 hrs. The mixture was cooled down and 15 ml of 2N NaOH was added. The product crashed out and was isolated by filtration. The solid was purified by column (SiOH) with DCM:EtOH:NH₃ 400-200:8:1 and isolated as a yellow solid (258 mg, 75%).

¹H NMR (D⁶-DMSO) δ 10.06 (1H, broad s), 9.29 (1H, s), 8.81 (1H, s) 8.63 (1H, s), 8.25 (2H, m), 8.09 (1H, s), 8.05 (1H, s), 7.83 (4H, m), 7.70 (1H, d, J 10 Hz), 7.39 (1H, t, J 10 Hz), 4.21 (2H, t, J 7.5 Hz), 2.68 (2H, t, J 7.5 Hz), 2.24 (6H, s).

LC-MS rt 1.99 m/z 470 ES+

### Example 21

### (6-Bromo-quinazolin-4-yl)-(4-[1,2,4]triazol-1-yl-phenyl)-amine

A stirred solution of N′-(4-bromo-2-cyano-phenyl)-N,N-dimethyl-formamidine (0.5 g, 2 mmol, 1 eq) and 4-[1,2,4]triazol-1-yl-phenylamine (0.32 g, 2 mmol, 1 eq) in acetic acid (4 mL) was heated to reflux for 2 hours then allowed to cool. After cooling, addition of diethylether afforded a yellow precipitate. The precipitate was collected by filtration, washed with diethylether then dried in vacuo to afford the desired compound as the acetate salt. (0.53 g, 62%). ¹H-NMR (DMSO-d₆) δ 1.88 (s, 3H), 7.77 (d, 1H), 7.90 (d, 2H), 8.02 (dd, 1H), 8.08 (d, 2H), 8.25 (s, 1H), 8.68 (s, 1H), 8.90 (d, 1H), 9.29 (dd, 1H), 10.04 (s, 1H). LC-MS rt 2.30 m/z 368 ES+.

### Example 22

### N-(2-{2-fluoro-4-[4-(4-[1,2,4]-triazol-1-yl-phenylamino)quinazoline-6-yl}ethyl)-N′,N′,N′-trimethylethane-1,2-diamine

A solution of Intermediate 11 (96 mg, 0.23 mmol, 1 eq), and 4-(1H-1,2,4-triazol-1-yl)aniline (38 mg, 0.24 mmol, 1.05 mmol) in acetic acid (2 mL) was heated at reflux. After 1 h the reaction was allowed to cool to room temperature, concentrated in vacuo and treated with a saturated aqueous solution of K₂CO₃ until effervescence ceased. The resulting mixture was then concentrated in vacuo to dryness to yield a brown residue. The residue was purified using flash column chromatography, eluting initially with 200:8:1 and then 100:8:1 CH₂Cl₂:EtOH:NH₃. The title compound was isolated as an off-white solid (48 mg, 40%). R_{f}=0.39 (40:8:1 CH₂Cl₂:EtOH:NH₃). ¹H-NMR (DMSO-d₆) 2.11 (s, 6H), 2.17 (s, 3H), 2.43 (m, 2H), 2.68 (t, 2H), 4.08 (t, 2H), 7.23 (m, 1H), 7.56 (m, 1H), 7.75 (m, 3H), 7.93 (m, 2H), 8.08 (m, 2H), 8.50 (s, 1H), 8.68 (s, 1H), 9.14 (s, 1H), 9.92 (br. s, 1H). LC-MS rt 2.08 m/z 527 ES+

### Example 23

### [6-(3-Isopropoxy-4-methoxy-phenyl)-quinazolin-4-yl]-(4-[1,2,4]triazol-1-yl-phenyl)-amine

To a solution of Intermediate 18 (60 mg, 0.18 mmol) in acetic acid (1 ml) was added 4-triazolylaniline (35 mg, 0.22 mmol) and the solution was stirred at 80° C. over 2 hours. LCMS analysis showed consumption of starting material.
Reaction mixture was cooled and the excess acetic acid removed by evaporation under reduced pressure. The residues were treated with sat. aq. Sodium hydrogen carbonate until cessation of effervescence. The resulting precipitate was filtered and the solids obtained dried under vacuum.
Purification by preparative chromatography furnished the desired compound as an off-white solid (18 mg, 22%)

¹H NMR (D⁶-DMSO) 10.21 (1H, bs), 9.07 (1H, s), 8.57 (1H, s), 8.42 (1H, s), 8.04 (1H, s), 8.00 (1H, dd, J 8.75 Hz, 1.5 Hz), 7.90 (2H, d, J 9 Hz), 7.72 (1H, s), 7.68 (2H, d, J=3.25 Hz), 7.27 (2H, s), 6.96 (1H, d, J 9 Hz), 4.53 (1H, quin, J 6 Hz), 3.64 (3H, s), 1.12 (6H, d, J 6.25 Hz)

LC-MS rt 2.48 m/z 454 ES+

### Example 24

### [6-(4-Fluoro-3-isopropoxy-phenyl)-quinazolin-4-yl]-(4-[1,2,4]triazol-1-yl-phenyl)-amine

To a solution of Intermediate 19 (95 mg, 0.29 mmol) in acetic acid (1 ml) was added 4-triazolylaniline (56 mg, 0.35 mmol) and the solution was stirred at 80° C. over 2 hours. LCMS analysis showed consumption of starting material.
Reaction mixture was cooled and the excess acetic acid removed by evaporation under reduced pressure. The residues were treated with sat. aq. Sodium hydrogen carbonate until cessation of effervescence. The resulting precipitate was filtered and the solids obtained dried under vacuum.
Purification by preparative chromatography furnished the desired compound as an off-white solid (33 mg, 25%)

¹H NMR (D⁶-DMSO) 10.24 (1H, bs), 9.27 (1H, s), 8.82 (1H, s), 8.64 (1H, s), 8.36 (1H, s), 8.24 (1H, s), 8.22 (1H, dd, J 9 Hz, 1.75 Hz), 8.09 (2H, d, J 9 Hz), 7.92 (3H, m), 7.64 (1H, dd, J 8.25 Hz, 2 Hz), 7.46 (1H, m), 4.87 (1H, quin, J 6 Hz), 1.36 (6H, d, J 6 Hz)

LC-MS rt 2.66 m/z 441 ES+

### Example 25

### {6-[4-(2-Dimethylamino-ethoxy)-3-fluoro-phenyl]-quinazoline-4-yl}-(4-[1,2,4]triazole-1-yl-phenyl)amine

### Step 1: 4-Bromo-1-(3-chloro-propoxy)-2-fluoro-benzene

A mixture of 2-fluoro-4-bromophenol (162.6 mmol, 31.07 g) powdered potassium carbonate (731.7 mmol, 103 g), 1-bromo-3-chloropropane (270 mmol, 26.6 ml) in acetonitrile (250 ml) was heated to reflux for 3 h. The cooled reaction mixture was filtered through celite and concentrated under vacuum. This gave a pale oil (43.5 g, 100%).

¹H NMR (CDCl₃) δ 6.99 (2H, t, J 8.75 Hz), 6.67 (1H, t, J 8.75 Hz), 3.97 (2H, t, J 6.00 Hz), 3.57 (2H, t, J 6.00 Hz), 2.04 (2H, m).

### Step 2: 1-[3-(4-bromo-2-fluoro-phenoxy)-propyl]-pyrrolidine

A mixture of 4-Bromo-1-(3-chloro-propoxy)-2-fluoro-benzene from step 1 (0.046 mM, 11.94 g) and pyrrolidine (0.138 mM, 11.43 ml) in DMA (50 ml) was heated to reflux for 48 h. The cooled reaction mixture was partitioned between ethylacetate and saturated NaHCO₃. The combined organic phases were dried over magnesium sulphate, concentrated and a brown oil was isolated (13.5 g, 98%).

¹H NMR (CDCl₃) δ 7.11 (2H, m), 6.79 (1H, t, J 8.75 Hz), 4.01 (2H, t, J 6.25 Hz), 2.52 (2H, t, J 7 Hz), 2.42 (6H, m), 1.95 (3H, m), 1.69 (1H, m, obscured by H2O).

### Step 3: 3-Fluoro-4-(3-pyrrolidine-1-yl-propoxy)-phenylboronic acid

A solution of 1-[3-(4-bromo-2-fluoro-phenoxy)-propyl]pyrrolidine from step 2 (5.31 g, 17.57 mmol) in THF (10 ml) was added drop-wise to a stirred slurry of Mg (640 mg, 1.5 eq) in THF (2 ml) and a small crystal of iodine. After addition, the mixture was heated to reflux for 3 h. The grey mixture was cooled to −78° trimethylborate (1.2 eq, 2.4 ml) added drop-wise and allowed to warm overnight. 2N HCl (aq, 60 ml) was added and stirred for 30 min before being extracted into ether (2×50 ml). The combined organic phases were dried, concentrated and the resulting brown oil triturated with acetone/ether/petrol, to give a brown solid. The product was used crude without further purification.

¹H NMR (d₅-DMSO) δ 8.08 (2H, broad s), 7.55 (1H, t, J 8.75 Hz), 7.15 (2H, m), 4.04 (2H, t, J 6.25 Hz), 2.50 (3H, m), 2.45 (6H, m), 1.84 (3H, m).

### Step 4: N′-[3-Cyano-3′-fluoro-4′-(3-pyrrolidin-1-yl-propoxy)-biphenyl-4-yl]-N,N-dimethyl-formamidine

A mixture of 3-fluoro-4-(3-pyrrolidine-1-yl-propoxy)-phenylboronic acid (3.074 g, 2 eq), intermediate 2 (1.72 g, 1 eq), potassium carbonate (0.954 g, 1.2 eq) in DMF/H₂O (3:1, 40 ml) was treated with dichloro(bis benzonitrile) palladium (II) (2%, 44 mg) and stirred at ambient under N₂ for 18 h. The mixture was diluted with water (200 ml) and extracted with ethyl acetate (2×50 ml). After drying over magnesium sulphate these organics phases were concentrated to a brown gum.

The residue was dissolved in DCM, loaded onto a short column of silica and eluted portion-wise under suction with DCM/EtOH/NH₃ 200:8:1 to 50:8:1 to give after concentration a light brown solid (1.63 g, 71%).

LC-MS rt 1.99 m/z 395 ES+

### Step 5: {6-[3 fluoro-4-(3-pyrrolidin-1-yl-propoxy)-phenyl]-quinazoline-4-yl}-(4-[1,2,4]triazole-1-yl-phenyl)amine

To a solution of formamidine from Step 4 (257 mg, 0.653 mM) in acetic acid (3 ml) was added the 4-triazolylaniline (115 mg, 0.73 mM). The reaction was heated to 125° C. for 1 h. The mixture was cool down basified with 15 ml of 2N NaOH and extracted with ethylacetate. After drying over magnesium sulphate these organics phases were concentrated to a yellow solid. The solid was triturated with DCM/Et2O/petrol and isolated as a yellow solid (163 mg, 44%).

¹H NMR (D₆-DMSO) δ 10.07 (1H, broad s), 9.29 (1H, s), 8.83 (1H, s), 8.64 (1H, s) 8.26 (2H, m), 8.07 (2H, m), 7.85 (5H, m), 7.69 (1H, d, J 10 Hz), 7.35 (1H, dd, J 10 Hz, 5 Hz), 4.22 (2H, t, J 7.5 Hz), 2.46 (6H, m, obscured by H2O), 2.08 (2H, t, J 7.5 Hz), 1.70 (4H, m).

LC-MS rt 2.13 m/z 509 ES+

### Example 26

### {6-[3-fluoro-4-(3-morpholin-4-yl-propoxy)-phenyl]-quinazoline-4-yl}-(4-[1,2,4]triazole-1-yl-phenyl)amine

### Step 1: 4-Bromo-1-(3-chloro-propoxy)-2-fluoro-benzene

A mixture of 2-fluoro-4-bromophenol (162.6 mmol, 31.07 g), powdered potassium carbonate (731.7 mmol, 103 g), 1-bromo-3-chloropropane (270 mmol, 26.6 ml) in acetonitrile (250 ml) was heated to reflux for 3 h. The cooled reaction mixture was filtered through celite and concentrated under vacuum. This gave a pale oil (43.5 g, 100%).

¹H NMR (CDCl₃) δ 6.99 (2H, t, J 8.75 Hz), 6.67 (1H, t, J 8.75 Hz), 3.97 (2H, t, J 6.00 Hz), 3.57 (2H, t, J 6.00 Hz), 2.04 (2H, m).

### Step 2: 4-[3-(4-bromo-2-fluoro-phenoxy)-propyl]-morpholine

A mixture of 4-Bromo-1-(3-chloro-propoxy)-2-fluoro-benzene from step 1 (0.038 mM, 10.02 g) and morpholine (0.114 mM, 10.1 ml) in DMA (50 ml) was heated to reflux for 48 h. The cooled reaction mixture was partitioned between ethyl acetate and saturated NaHCO₃. The combined organic phases were dried over magnesium sulphate, concentrated and a brown oil was isolated (12 g, 98%).

¹H NMR (CDCl₃) δ 7.36 (1H, m), 7.32 (1H, m), 6.98 (1H, t, J 8.75 Hz), 4.24 (2H, t, J 6.25 Hz), 3.87 (4H, m), 2.62 (6H, m), 2.12 (2H, m).

LC-MS rt 1.98 m/z 320 ES+

### Step 3: 3-Fluoro-4-(3-morpholine-1-yl-propoxy)-phenylboronic acid

A solution of 4-[3-(4-bromo-2-fluoro-phenoxy)-propyl]-morpholine from step 2 (5.08 g, 15.96 mmol) in THF (10 ml) was added drop-wise to a stirred slurry of Mg (582 mg, 1.5 eq) in THF (2 ml) and a small crystal of iodine. After addition, the mixture was heated to 90° for 1 h. The grey mixture was cooled to −78° trimethylborate (1.2 eq, 2.15 ml) added drop-wise and allowed to warm overnight. 2N HCl (aq, 60 ml) was added and stirred for 30 min before being extracted into ether (2×50 ml). Then triethylamine was added to the aqueous layer and extracted with ethyl acetate and ether. The combined organic phases were dried, concentrated to give a brown oil (3.8 g, 84%). The product was used crude without further purification.

¹H NMR (D₆-DMSO) δ 8.08 (2H, broad s), 7.55 (1H, t, J 8.75 Hz), 7.15 (2H, m), 4.04 (2H, t, J 6.25 Hz), 3.57 (4H, m), 2.37 (6H, m), 1.84 (2H, m).

LC-MS: rt 1.95 m/z 284 ES+.

### Step 4: N′-[3-Cyano-3′-fluoro-4′-(3-morpholin-1-yl-propoxy)-biphenyl-4-yl]-N,N-dimethyl-formamidine

A mixture of 3-fluoro-4-(3-morpholine-1-yl-propoxy)-phenylboronic acid (3.8 g, 1.5 eq), intermediate 2 (2.67 g, 1 eq), potassium carbonate (1.48 g, 1.2 eq) in DMF/H₂O (3:1, 40 ml) was treated with dichloro(bis benzonitrile) palladium (II) (2%, 68 mg) and stirred at ambient under N₂ for 18 h. The mixture was diluted with water (200 ml) and extracted with EtOAc (2×50 ml). After drying over magnesium sulphate these organics phases were concentrated to a brown gum.
The residue was dissolved in DCM, loaded onto a short column of silica and eluted portion-wise under suction with DCM/EtOH/NH₃ 200:8:1 to give after concentration a mobile brown solid (3.16 g, 85%). The product solidified on standing.

¹H NMR (CDCl₃) δ 7.79 (1H, broad s), 7.42 (1H, m), 7.02 (1H, m), 6.79 (3H, m) 3.88 (2H, m), 3.49 (4H, m), 2.87 (2H, d, J 5.5 Hz), 2.73 (3H, s), 2.66 (3H, s), 2.30 (5H, m), 1.75 (2H, m).

LC-MS rt 1.91 m/z 411 ES+

### Step 5: {6-[3-fluoro-4-(3-morpholin-4-yl-propoxy)-phenyl]-quinazoline-4-yl}-(4-[1,2,4]triazole-1-yl-phenyl)amine

To a solution of formamidine from Step 4 (429 mg, 1.045 mmol) in acetic acid (3 ml) was added the 4-triazolylaniline (184 mg, 1.15 mmol). The reaction was heated to 125° C. for 1 h. The mixture was cooled down basified with 15 ml of 2N NaOH and the resulting precipitate isolated by filtration then dissolved in DCM and purified by column chromatography on silica with DCM/EtOH/NH₃ 400:8:1 up to 50:8:1 to give after concentration a cream solid (136 mg, 25%).

LC-MS rt 2.1 m/z 526 ES+.

¹H NMR (D₆-DMSO) δ 10.06 (1H, broad s), 9.29 (1H, s), 8.82 (1H, s), 8.65 (1H, s) 8.21 (2H, m), 8.07 (2H, m), 7.83 (4H, m), 7.69 (1H, d, J 10 Hz), 7.32 (1H, dd, J 10 Hz, 5 Hz), 4.17 (2H, t, J 7.5 Hz), 3.58 (4H, t, J 2.5 Hz), 3.34 (2H, m, obscured by H2O), 2.40 (4H, t, J 2.5 Hz), 1.92 (2H, t, J 7.5 Hz).

### Example 27

### [6-(3,4-Dimethoxy-phenyl)-quinazolin-4-yl]-(2-methyl-4-[1,2,4]triazol-1-yl-phenyl)-amine

To a carousel tube was added the formamidine (example 10 step 1) (0.05 g, 0.16 mmol), Intermediate 21 (0.031 g, 0.18 mmol) and AcOH (1.5 ml). The mixture was left to stir at 120° C. for 3 hr. The mixture was allowed to cool and concentrated to dryness and put on a silica column and eluted with 2.5% MeOH: DCM. A white solid was isolated 0.015 g, (21%).

¹H n.m.r (D₆-DMSO) 9.69 (1H, s), 9.10 (1H, s), 8.54 (1H, s), 8.23 (1H, s), 8.06 (1H, s), 8.02 (1H, m), 7.69-7.24 (6H, m), 6.94 (1H, d, J=8.85 Hz), 3.71 (3H, s), 3.64 (3H, s), 2.11 (3H, s);

LC-MS rt 2.18 m/z 438 ES+

### Example 28

### 1-(4-{6-[3-Fluoro-4-(2-methoxy-ethoxy)-phenyl]-quinazolin-4-ylamino}-phenyl)-5-methyl-1H-[1,2,4]triazole-3-carboxylic acid

To a solution of Intermediate 22 (55 mg, 0.16 mM) in acetic acid (3 ml) was added the intermediate 24 (42 mg, 0.19 mM). The reaction was heated to 100° C. for 2 hrs. The mixture was cooled down and 15 ml of 2N NaOH was added. The product crashed out and was isolated by filtration. The solid was purified by preparative HPLC and isolated as a white solid (26 mg, 32%).

¹H NMR (D₆-DMSO) δ 10.15 (1H, broad s), 8.82 (1H, s), 8.56 (1H, s) 8.15 (1H, d, J 7.5 Hz), 8.00 (2H, d, J 7.5 Hz), 7.80 (2H, m), 7.68 (1H, d, J 7.5 Hz), 7.56 (2H, d, J 7.5 Hz), 7.32 (1H, t, J 7.5 Hz), 4.27 (3H, s), 3.73 (3H, s); LC-MS: it 2.31 m/z 512 ES⁻.

### Example 29

### 1-(4-{6-[3,4-Dimethoxy-phenyl]-quinazolin-4-ylamino}-phenyl)-5-methyl-1H-[1,2,4]triazole-3-carboxylic acid

To a solution of Intermediate 23 (50 mg, 0.16 mM) in acetic acid (3 ml) was added the Intermediate 24 (42 mg, 0.19 mM). The reaction was heated to 100° C. for 2 hrs. The mixture was cooled down and 15 ml of 2N NaOH was added. The product crashed out and was isolated by filtration. The solid was purified by preparative HPLC and isolated as a white solid (27 mg, 34%).

¹H NMR (D₆-DMSO) δ 10.11 (1H, broad s), 8.82 (1H, s), 8.68 (1H, s) 8.26 (1H, dd, J 10 Hz, J 2.5 Hz), 8.14 (2H, d, J 10 Hz), 7.87 (1H, d, J 10 Hz), 7.67 (2H, d, J 10 Hz), 7.46 (2H, dd, J 10 Hz, J 2.5 Hz), 7.14 (1H, d, J 10 Hz), 3.93 (3H, s), 3.85 (3H, s); LC-MS: rt 2.16 m/z 482 ES⁺.

### Example 30

### 1-{-4-[6-(3,4-Dimethoxy-phenyl)-quinazolin-4-ylamino]-phenyl}-5-methyl-1H-[1,2,4]triazole-3-carboxylic acid dimethylamide

To a solution of Intermediate 23 (50 mg, 0.16 mM) in acetic acid (3 ml) was added Intermediate 25 (49 mg, 0.19 mM). The reaction was heated 2 hrs to 100° C. The mixture was cooled down and 15 ml of 2N NaOH was added. The product crashed out and the solid was collected by filtration. The solid was purified by preparative HPLC and isolated as a white solid (23 mg, 28%).

¹H NMR (D₆-DMSO) δ 10.09 (1H, broad s), 8.82 (1H, s), 8.67 (1H, s) 8.26 (1H, dd, J 8.75 Hz, J 1.5 Hz), 8.13 (2H, d, J 8.75 Hz), 7.87 (1H, d, J 8.7 Hz), 7.66 (2H, d, J 8.7 Hz), 7.46 (2H, m), 7.14 (1H, d, J 8.7 Hz), 3.93 (3H, s), 3.85 (3H, s), 3.15 (3H, s), 3.03 (3H, s), 2.55 (3H, s); LC-MS: rt 2.22 m/z 510 ES⁺

### Example 31

### 1-(4-{6-[3-Fluoro-4-(2-methoxy-ethoxy)-phenyl]-quinazolin-4-ylamino}-phenyl)-5-methyl-1H-[1,2,4]triazole-3-carboxylic acid methylamide

### Step 1: 5-Methyl-1-(4-nitrophenyl)-1H-[1,2,4]triazole-3-carboxylic acid methylamide

To a solution of 5-Methyl-1-(4-nitrophenyl)-1H-1,2,4-triazole-3-carboxylic acid (Key Organics, 400 mg, 1.61 mM) and Hunig's base (667 ul, 3.86 mM) in DCM:DMF (6 ml:1 ml) at −10° C. was added drop-wise isobutylchloroformate (250 ul, 1.93 mM). The reaction was stirred 30 min. at −10° C. then slowly a 2M solution of methylamine (965 ul, 1.93 mM) was added. The mixture was allowed to warm-up to r.t. and stirred for another hour. The crude mixture was washed with sat. NaHCO₃ extracted and dried over MgSO₄. After evaporation the solid obtained was used directly in the next reaction without further purification (374 mg, 89%). LC-MS: rt 2.01 m/z 261 ES⁺.

¹H NMR (d-DMSO) δ 8.42 (2H, d, J 7 Hz), 7.95 (2H, d, J 7 Hz), 2.73 (3H, d, J 4.75 Hz), 2.60 (3H, s).

### Step 2: 1-(4-Amino-phenyl)-5-methyl-1H-[1,2,4]triazole-3-carboxylic acid methylamide

A solution of 5-Methyl-1-(4-nitrophenyl)-1H-[1,2,4]triazole-3-carboxylic acid methylamide from step 1 (100 mg, 0.38 mmol) in methanol (8 ml) was injected at a 1 ml/min rate into an hydrogenator “the H-Cube” that combines endogenous hydrogen generation with a disposable cartridge system (Pd/C), temperature was set up to 25° C. at atmospheric pressure.
The solution obtained was concentrated to give a white solid (86 mg, 97%). LC-MS: rt 1.15 m/z 231 ES⁺.

¹H NMR (d-DMSO) δ 8.61 (1H, broad s), 8.54 (2H, d, J 7 Hz), 7.12 (2H, d, J 7 Hz), 2.73 (3H, d, J 4.75 Hz), 2.60 (3H, s).

### Step 3: 1-(4-{6-[3-Fluoro-4-(2-methoxy-ethoxy)-phenyl]-quinazolin-4-ylamino}-phenyl)-5-methyl-1H-[1,2,4]triazole-3-carboxylic acid methylamide

To a solution of Intermediate 22 (50 mg, 0.16 mM) in acetic acid (3 ml) was added 1-(4-Amino-phenyl)-5-methyl-1H-[1,2,4]triazole-3-carboxylic acid methylamide from step 2 (44 mg, 0.19 mM). The reaction was stirred 2 hrs at a 100° C. The mixture was cooled down and 15 ml of 2N NaOH was added. The product crashed out and the solid was collected by filtration. The solid was purified by preparative HPLC and isolated as a white solid (6 mg, 8%). LC-MS: rt 2.33 m/z 528 ES⁺.

¹H NMR (d-DMSO) δ 10.02 (1H, broad s), 8.74 (1H, s), 8.56 (1H, s) 8.21 (1H, dd, J 8.75 Hz, J 1.5 Hz), 8.01 (2H, d, J 8.7 Hz), 7.79 (2H, m), 7.60 (2H, d, J 8.7 Hz), 7.57 (1H, s), 7.27 (1H, t, J 8.7 Hz), 4.15 (2H, t, J 4.5 Hz), 3.62 (2H, t, J 4.5 Hz), 3.24 (3H, s), 2.67 (3H, d, J 4.7 Hz), 2.44 (3H, s).

### Example 32

### 1-(4-{6-[3-Fluoro-4-(2-methoxy-ethoxy)-phenyl]-quinazolin-4-ylamino}-phenyl)-5-methyl-1H-[1,2,4]triazole-3-carboxylic acid dimethylamide

To a solution of Intermediate 22 (50 mg, 0.16 mM) in acetic acid (3 ml) was added the intermediate 25 (47 mg, 0.19 mM). The reaction was heated 2 hrs to 100° C. The mixture was cooled down and 15 ml of 2N NaOH was added. The product crashed out and the solid was collected by filtration. The solid was purified by preparative HPLC and isolated as a white solid (23 mg, 27%).

¹H NMR (d-DMSO) δ 10.09 (1H, broad s), 8.83 (1H, s), 8.66 (1H, s) 8.22 (1H, dd, J 7.5 Hz, J 1.5 Hz), 8.14 (1H, d, J 5 Hz), 8.10 (1H, s), 7.85 (2H, m), 7.70 (2H, s), 7.66 (1H, s), 7.33 (1H, dd, J 10 Hz, J 7.5 Hz), 4.25 (2H, t, J 5 Hz), 3.71 (2H, t, J 5 Hz), 3.14 (3H, s), 3.01 (3H, s), 2.67 (3H, s), 2.44 (3H, s); LC-MS: rt 2.37 m/z 542 ES⁺.

### Example 33

### (6-Bromo-quinazolin-4-yl)-(2-methyl-4-[1,2,4]triazol-1-yl-phenyl-amine

To a carousel tube was added 6-bromo-4-chloro-quinazoline (0.1 g, 0.41 mmol), Intermediate Y (0.078 g, 0.45 mmol) and CH₃CN (anhydrous, 4 ml) and the mixture was stirred at 90° C. under nitrogen for 24 hr. A orange precipitate had formed which was filtered off and washed with water, 1N NaOH and water again and dried under a vacuum at 40° C. Isolated 0.1 g (51%) of solid.

¹H n.m.r (D₆-DMSO) 9.26 (1H, s), 9.05 (1H, s), 8.76 (1H, s), 8.20 (1H, s), 8.17 (1H, d, J=1.26 Hz), 7.85 (2H, bs), 7.81 (1H, s), 7.74 (1H, dd, J=8.85 Hz, 2.53 Hz), 7.47 (1H, d, J=8.21 Hz), 2.25 (3H, s); LC-MS rt 2.19 m/z 382 ES+

### Example 34

### [1-(4-{6-[3-Fluoro-4-(2-methoxy-ethoxy)-phenyl]-quinazolin-4-ylamino}-phenyl)-5-methyl-1H-[1,2,4]triazole-3-yl]-(4-methyl-piperazin-1-yl)methanone

### Step 1: [5-Methyl-1-(4-nitro-phenyl)-1H-[1,2,4]triazol-3-yl]-(4-methyl-piperazin-1-yl)-methanone

To a solution of 5-Methyl-1-(4-nitrophenyl)-1H-1,2,4-triazole-3-carboxylic acid (Key Organics, 400 mg, 1.61 mM) and Hunig's base (667 ul, 3.86 mM) in DCM:DMF (6 ml:1 ml) at −10° C. was added drop-wise isobutylchloroformate (250 ul, 1.93 mM). The reaction was stirred 30 min. at −10° C. then slowly methylpiperazine (214 ul, 1.93 mM) was added. The mixture was allowed to warm-up to r.t. and stirred for another hour. The crude mixture was washed with sat. NaHCO₃ extracted and dried over MgSO₄. After evaporation the solid obtained was used directly in the next reaction without further purification (335 mg, 63%). LC-MS: rt 1.26 m/z 330 ES⁺.

¹H NMR (D₆-DMSO) δ 8.39 (2H, d, J 7 Hz), 7.92 ((2H, d, J 7 Hz), 3.58 (4H, m), 3.30 (4H, m), 2.58 (3H, s), 2.15 (3H, s).

### Step 2: [1-(4-Amino-phenyl)-5-methyl-1H-[1,2,4]triazol-3-yl]-(4-methyl-piperazin-1-yl)methanone

A solution of [5-Methyl-1-(4-nitro-phenyl-)-1H-[1,2,4]triazol-3-yl]-(4-methyl-piperazin-1-yl)-methanone from step 1 (100 mg, 0.302 mmol) in methanol (8 ml) was injected at a 1 ml/min rate into an hydrogenator “the H-Cube” that combines endogenous hydrogen generation with a disposable cartridge system (Pd/C), temperature was set up to 25° C. at atmospheric pressure.
The solution obtained was concentrated to give a white solid (87 mg, 96%). LC-MS: rt 2.22 m/z 301 ES⁺.

¹H NMR (D₆-DMSO) δ 8.60 (1H, broad s), 7.51 (2H, d, J 7 Hz), 7.09 ((2H, d, J 7 Hz), 3.58 (4H, m), 3.30 (4H, m), 2.58 (3H, s), 2.15 (3H, s).

### Step 3: [1-(4-{6-[3-Fluoro-4-(2-methoxy-ethoxy)-phenyl]quinazolin-4-ylamino}-phenyl)-5-methyl-1H-[1,2,4]triazole-3-yl]-(4-methyl-piperazin-1-yl)methanone

To a solution of Intermediate 22 (55 mg, 0.16 mM) in acetic acid (3 ml) was added [1-(4-Amino-phenyl)-5-methyl-1H-[1,2,4]-triazol-3-yl]-(4-methyl-piperazin-1-yl)methanone from step 2 (58 mg, 0.19 mM). The reaction was heated 2 hrs to 100° C. The mixture was cooled down and 15 ml of 2N NaOH was added. The product crashed out and the solid was collected by filtration. The solid was purified by preparative HPLC and isolated as a white solid (25 mg, 26%). LC-MS: rt 2.11 m/z 597 ES⁺.

¹H NMR (D₆-DMSO) δ 10.32 (1H, broad s), 9.05 (1H, s), 8.88 (1H, s) 8.45 (1H, dd, J 7.5 Hz, J 1.5 Hz), 8.35 (1H, d, J 5 Hz), 8.31 (1H, s), 8.08 (2H, m), 7.92 (2H, s), 7.86 (1H, s), 7.58 (1H, dd, J 10 Hz, J 7.5 Hz), 4.47 (2H, t, J 5 Hz), 3.95 (2H, t, J 5 Hz), 3.88 (4H, m), 3.57 (3H, obscured by H₂O), 3.53 (4H, obscured by H₂O), 2.78 (3H, s), 2.43 (3H, s).

### Example 35

### 2-Methoxy-4-[4-(4-[1,2,4]triazol-1-yl-phenylamino)-quinazolin-6-yl]-phenol

### Step 1: N′-(3-Cyano-4′-hydroxy-3′-methoxy-biphenyl-4-yl)-N,N-dimethyl-formamidine

A mixture of boronate (Aldrich, 2-methoxy-4-(4,4,5,5-tetramethyl-1,3,2-dioxaborolan-2-yl)phenol, 500 mg, 1.2 eq) and Intermediate 2 (1 eq, 498 mg), potassium carbonate (1.2 eq, 276 mg) in DMF/H2O (3:1, 12 ml) was treated with Pd Cl₂(PhCN)₂ (2%, 13 mg) and stirred at rt under N2 for 12 h. The mixture was diluted with water (50 ml) and filtered through a plastic frit under suction. The filter cake was washed with diethyl; ether and dried in vacuo to give a wine coloured solid (390 mg, 79%),

¹H NMR (D₆-DMSO) δ 3.18 (6H, d); 3.98 (3H, s); 6.94 (1H, d), 7.2 (1H, d); 7.32 (3H, m); 7.88 (1H, d); 7.99 (1H, s); 8.11 (1H, s); 9.23 (1H, s); LCMS rt 1.93 m/z 295 ES+

### Step 2: 2-Methoxy-4-[4-(4-[1,2,4]triazol-1-yl-phenylamino)-quinazolin-6-yl]-phenol

A mixture of N′-(3-Cyano-4′-hydroxy-3′-methoxy-biphenyl-4-yl)-N,N-dimethyl-formamidine (220 mg) and triazolyl aniline (113 mg) in acetic acid (2 ml) was heated to 125 for 2 h. The cooled mixture was diluted with water and filtered under suction overnight. The resulting solid was sonicated in acetone (20 ml) with warming and filtered. The solid was dried to give the title compound as a yellow solid (28 mg).

¹H NMR (D₆-DMSO) δ 3.86 (3H, s); 6.88 (1H, d); 7.28 (1H, d); 7.36 (1H, s); 7.82 (3H, m); 8.03 (2H, m); 8.15 (2H, m); 8.56 (1H, s); 8.69 (1H, s); 9.2 (2H, s); 9.98 (1H, s); LC-MS rt 2.14 m/z 411 ES+

### Example 36

### 2-Methoxy-5-[4-(4-[1,2,4]triazol-1-yl-phenylamino)-quinazolin-6-yl]-phenol

This compound may be prepared by the method in Example 35, the appropriate boronate being prepared from 5-bromo-2-methoxyphenol, itself prepared by Sandmeyer reaction (see JOC, 1993, 58, 1, 42). The required boronic acid/boronate may be prepared by a palladium catalysed boronation using bis(pinacolato)borane or by temporary protection of the phenol ie with dihydropyran, followed by lithiation, reaction with trimethoxyborate followed hydrolysis and concomitant removal of the protecting group.

### Example 37

### {6-[3-Fluoro-4-(2-methoxy-ethoxy)-phenyl]-quinazolin-4-yl}-(2-methyl-4-[1,2,4]triazole-1-yl-phenyl)-amine

This compound may be prepared by the method outlined in Example 27, using the appropriate formamidine described as intermediate 22.

### Example 38

### HCV Replicon Activity

### Materials:

HCV replicon cell line

1b replicon (Huh.7) described in Science 285, 110-113.
Huh-9B: liver cell line with persistent bicistronic HCV genotype 1b coding sequence: [I₃₈₉1ucubineo_{—}3-3′_ET] includes firefly luciferase-ubiquitin-neomycin phosphotransferase fusion protein and EMCV-IRES driven non-structural HCV (NS3 to NS5B) coding sequence including cell culture adaptive mutations E1202G, T12801 and K1846T (Lohmann et al, 2001).

### Method:

This assay is set up using all 96 wells of flat-bottomed 96-well plates. Plates are set up one day before addition of compounds. The assay then runs for 4 days with ELISA development taking place on the 5^{th} day.

### Day 1

### Set up of Assay Plates

Exponentially growing Huh-9B monolayers are washed with sterile PBS to remove serum and treated with trypsin to detach cells from the flask.

Cells are suspended in growth media and counted using a haemocytometer. Duplicate 96 well plates are seeded with Huh-9B at a density of 10⁴ cells/well in a total volume of 100 μl/well of growth medium without antibiotics as depicted below.

One of the plates is an opaque white 96-well plate used for IC50 determination based on the luciferase signal (referred as replicon plate), the other one is a clear 96-well plate used for a parallel determination of drug toxicity by methylene blue staining (referred as tox plate). Wells G12 and H12 of the tox plate are left without cells to use as buffer alone background reading.

Plates are then incubated at 37° C. in a 5% CO₂ environment for 24 h to obtain a 90% confluent cell monolayer.

### Day 2

### Addition of Compound and Compound Dilutions

Doubling dilutions of each compound are generated in a separate 96 high volume capacity round bottom plate to twice their initial concentration in the assay using growth medium without antibiotics.

Five compounds (C1 to C5) are tested on each assay plate as illustrated below plus a control compound that is also included in each plate.

Compounds are tested across an 8 point doubling dilution series. The initial dilution of each compound to be tested is 25 μM and 12.5 μM for the control compound.

DMSO only wells (A1 and A2) at 1% provide the signal corresponding to maximal (100%) luciferase detection. Previous optimization experiments showed negligible luciferase signal from non-replicon containing cells and control wells for background (unspecific) level of detection are not routinely included. The signal from the DMSO wells at 1% (maximal signal) constitutes the assay window.

For each compound dilution on the 2×96-well dilution plate 100 μl are transferred using a multichannel pipette onto the replicon and tox plates mirror wells which contain 100 p. 1 of medium to obtain the desired final concentration.

### Day 5

### Luciferase Detection Stage on the Replicon Plate

Media is tapped out from wells into Virkon and plates are washed once in warm PBS and tapped dry gently.

For each well 20 μl of lysis buffer is added by multichannel pipette. Lysates are stable at this point for several hours.

Luciferase assay buffer is placed it in the luminometer (Lmax, Molecular Devices).

The M injector is primed with 4×300 μl of luciferase assay buffer. The plate to be analyzed is placed in the luminometer and 100 p. 1 of luciferase assay buffer injected automatically into one well followed by 4 seconds integration read out.

After one second delay a second well is injected with 100 μl of luciferase assay buffer followed by 4 seconds integration read out and so forth until all 96 wells are analyzed.

Once the reading is finished the luminometer injection system is washed with deionised water.

The data is acquired using the SOFTmax for Lmax Pro software package.

### Toxicity Determination on the Tox Plate

Media is tapped out from wells into Virkon and plates tapped dry gently. To each well 100 μl of 0.5% solution of methylene blue in 50% methanol is added to all wells including blanks (G12 and H12). Plates are left at RT for a minimum of 1 h. Plates are then rinsed gently by immersing in a plastic box with water, tapped dry gently and left open until they are fully dry. Dye is solubilized adding 100 μl of 1% lauroylsarcosine to each well and shaking for 1 h at 37° C. Plates are read on the SpectraMax spectrophotometer at 620 nm wavelength using the SOFTmax Pro software package.

### Results:

SOFTmax data files are exported as Excel or text files. A standard four parameters non-linear regression analysis of the data obtained from each compound is then used to calculate the IC50.

In the above analysis all replicate wells are meaned. The % of control is then calculated for each concentration point as a percentage of the DMSO control wells.

| | Replicon IC50 | |
|---|---|---|
| | *** <5 μM; ** = 5-20 μM; | Replicon TD50 |
| | * >20 μM | ** >25 μM, * <25 μM |
| Example No. | uM | uM |
| 1 | *** | * |
| 2 | ** | ** |
| 3 | *** | ** |
| 4 | ** | ** |
| 5 | *** | ** |
| 6 | * | ** |
| 7 | *** | ** |
| 8 | *** | ** |
| 9 | *** | ** |
| 10 | *** | * |
| 11 | *** | ** |
| 12 | *** | * |
| 13 | ** | * |
| 14 | *** | * |
| 15 | *** | ** |
| 16 | *** | ** |
| 17 | *** | ** |
| 18 | *** | ** |
| 19 | *** | ** |
| 20 | *** | * |
| 21 | *** | ** |
| 22 | *** | * |
| 23 | *** | * |
| 24 | *** | ** |
| 25 | *** | * |
| 26 | *** | * |
| 27 | *** | * |
| 28 | ** | ** |
| 29 | ** | ** |
| 30 | *** | * |
| 31 | *** | ** |
| 32 | *** | * |
| 33 | *** | ** |
| 34 | *** | ** |
| 35 | | |
| 36 | | |
| 37 | | |

### Example 39

### Synergistic Action Between HCV Replication Inhibitors and Human Interferon αA

ELISA experiments were carried out on the combined effect of HCV replication inhibitor [6-(3,4-Dimethoxy-phenyl)-quinazolin-4-yl]-(4-[1,2,4]triazol-1-yl-phenyl)-amine with interferon αA (Sigma Hu-INF-α A order number 14276).

### ELISA Protocol as in Example 38

The IC50 for [6-(3,4-Dimethoxy-phenyl)-quinazolin-4-yl]-(4-[1,2,4]triazol-1-yl-phenyl)-amine was calculated for each background concentration of interferon αA. Similarly, the IC50 for interferon αA was calculated for each background concentration of [6-(3,4-Dimethoxy-phenyl)-quinazolin-4-yl]-(4-[1,2,4]triazol-1-yl-phenyl)-amine.
[6-(3,4-Dimethoxy-phenyl)-quinazolin-4-yl]-(4-[1,2,4]triazol-1-yl-phenyl)-amine had an ELISA IC50 of 0.2 μM against HCV replicon 1b.
Interferon αA had an ELISA IC50 of 6 u/ml against HCV replicon 1b.
In combination, at concentrations of
[6-(3,4-Dimethoxy-phenyl)-quinazolin-4-yl]-(4-[1,2,4]triazol-1-yl-phenyl)-amine below its IC50, the IC50 of interferon αA was reduced from 6 u/ml to at least 0.008 u/ml. At concentrations of interferon αA below its IC50, the IC50 of [6-(3,4-Dimethoxy-phenyl)-quinazolin-4-yl]-(4-[1,2,4]triazol-1-yl-phenyl)-amine is reduced from 0.2 μM to at least 0.067 μM.
The fractional inhibitory concentratin (FIC) can be used to identify a synergistic interaction.

$$FIC = \frac{Lowest IC 50 Cpd {A}^{COMBINATION}}{IC 50 Cpd {A}^{ALONE}} + \frac{Lowest IC 50 Cpd {B}^{COMBINATION}}{IC 50 Cpd {B}^{ALONE}}$$

where FIC value
<0.5 SYNERGY
0.5-1.0 ADDITION
1.0-2.0 INDIFFERENCE
>2.0 ANTAGONISM
   FIC=0.008 u/ml+0.067 M=0.336

6 u/ml 0.2 μM

1. A quinazoline derivative of formula (I), or a pharmaceutically acceptable salt thereof: 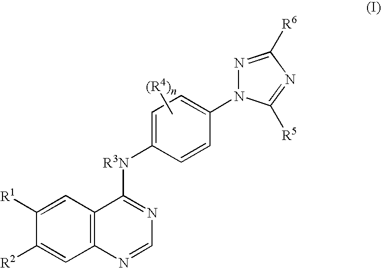
wherein:
R¹ represents halogen, C₁₋₆ alkyl, C₁₋₄ haloalkyl, C₁₋₆ alkoxy, C₁₋₄ haloalkoxy or a moiety -A, -A-A′, -A-Het-A′, -A-L-A′, -A-Het-L-A′, -A-L-Het-A′, -A-Het-L-Het′-A′ or -A-Het-L-Het′-L′;
R² represents hydrogen, halogen, C₁₋₄ alkyl, C₁₋₄ alkoxy, C₁₋₄ haloalkyl, C₁₋₄ haloalkoxy or a moiety -Het-A′, -Het-L-A′, -Het-L-Het′-A′ or -Het-L-Het′-L′;
R³ represents hydrogen or C₁₋₆ alkyl;
each R⁴ is the same or different and represents halogen C₁₋₄ alkyl, C₁₋₄ alkoxy or C₁₋₄ haloalkyl;
R⁵ represents hydrogen, C₁₋₄ alkyl, C₁₋₄ haloalkyl, C₁₋₄ alkoxy, C₁₋₄ aminoalkyl or a moiety -Het-L-Het′-L′;
R⁶ represents hydrogen, C₁₋₄ alkyl, C₁₋₄ haloalkyl, C₁₋₄ alkoxy, C₁₋₄ aminoalkyl or a moiety -Het-L-Het′-L′, —CO—X or -L-X where X represents —OR′, —NR′R″ or a 5- to 10-membered heteroaryl or heterocyclyl group wherein R′ and R″ are independently hydrogen or C₁₋₄ alkyl;
n is zero, 1 or 2;
each A and A′ are the same or different and represent a phenyl, 5- to 10-membered heteroaryl, 5- to 10-membered heterocyclyl or C₃₋₆ carbocyclyl group which is optionally fused to a further phenyl, 5- to 10-membered heteroaryl, 5- to 10-membered heterocyclyl or C₃₋₆ carbocyclyl group;
each Het and Het′ is the same or different and represents —O—, —S— or —NR′— where R′ is hydrogen or C₁₋₄ alkyl;
each L is the same or different and represents C₁₋₄ alkylene; and
each L′ is the same or different and represents hydrogen or a C₁₋₄ alkyl, C₁₋₄ haloalkyl, C₁₋₄ hydroxyalkyl or C₁₋₄ aminoalkyl group,
the phenyl, heteroaryl, heterocyclyl and carbocyclyl moieties in R¹, R² and R⁶ being unsubstituted or substituted by 1, 2 or 3 unsubstituted substituents which are the same or different and are selected from halogen atoms and C₁₋₄ alkyl, C₁₋₄ alkoxy, C₁₋₄ haloalkyl, C₁₋₄ haloalkoxy, hydroxy, cyano, nitro and —NR′R″, wherein each R′ and R″ is the same or different and represents hydrogen or C₁₋₄ alkyl.

2. (canceled)

3. A compound according to claim 1, wherein R¹ is halogen, C₁₋₄ alkyl, C₁₋₂ alkoxy, -A, -A-A′, -A-Het-L-A′ or -A-Het-L-Het′-L′.

4. (canceled)

5. A compound according to claim 1, wherein R² is hydrogen, halogen, C₁₋₄ alkyl, C₁₋₄ alkoxy, C₁₋₄ haloalkyl, C₁₋₄ haloalkoxy or a moiety -Het-L-A′ or -Het-L-Het′-L′.

6-10. (canceled)

11. A compound according to claim 1, wherein each A is the same or different and is a non-fused phenyl or 5- to 6-membered heteroaryl group or a phenyl fused to a further phenyl, 5- to 6-membered heteroaryl or 5- to 6-membered heterocyclyl group, said A group being unsubstituted or substituted with 1 or 2 unsubstituted substituents which are the same or different and are selected from halogen, hydroxy, C₁₋₄ alkyl and C₁₋₄ alkoxy.

12. A compound according to claim 1, wherein each A′ is the same or different and is a non-fused 5- to 6-membered heteroaryl or heterocyclyl group which is unsubstituted or substituted with 1 or 2 unsubstituted substituents which are the same or different and are selected from halogen, C₁₋₄ alkyl and C₁₋₂ alkoxy.

13-20. (canceled)

21. A compound according to claim 1, wherein:
R¹ is halogen, C₁₋₄ alkyl, C₁₋₂ alkoxy, -A, -A-A′, -A-O-L-A′ or -A-O-L-Het′-L′;
R² is hydrogen, C₁₋₄ haloalkoxy or a moiety —O-L-A′ or —O-L-Het′-L′;
R³ is hydrogen;
R⁴ is C₁₋₂ alkyl;
n is zero or 1;
R⁵ is hydrogen or C₁₋₄ alkyl;
R⁶ is hydrogen, C₁₋₄ alkyl, C₁₋₄ haloalkyl, C₁₋₄ alkoxy, C₁₋₄ aminoalkyl or —CO—X, wherein X is —OR′, —NR′R″ or a 5- to 6-membered heterocyclyl group which is unsubstituted or substituted by a C₁₋₂ alkyl group and R′ and R″ are the same or different and represent hydrogen or C₁₋₂ alkyl;
each A is the same or different and is a non-fused phenyl or 5- to 6-membered heteroaryl group or is a phenyl ring fused to a further phenyl, 5- to 6-membered heteroaryl or 5- to 6-membered heterocyclyl group, said A group being unsubstituted or substituted with 1 or 2 unsubstituted substituents which are the same or different and are selected from halogen, hydroxy, C₁₋₄ alkyl and C₁₋₄ alkoxy;
each A′ is the same or different and is an unsubstituted 5- to 6-membered heteroaryl or heterocyclyl group;
each Het′ is the same or different and is —O— or —NR′— where R′ is hydrogen or C₁₋₂ alkyl;
each L is the same or different is methylene, n-ethylene or n-propylene; and
each L″ is the same or different and is hydrogen, C₁₋₂ alkyl, C₁₋₂ haloalkyl, C₁₋₂ hydroxyalkyl or C₁₋₂ aminoalkyl.

22. (canceled)

23. A quinazoline derivative according to claim 1 having formula (Ib) or a pharmaceutically acceptable salt thereof: 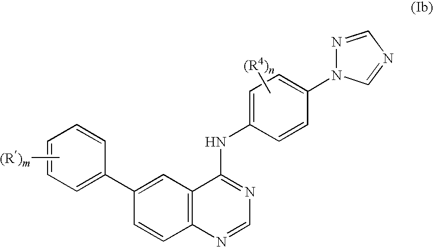
wherein:
each R⁴ is the same or different and represents halogen, C₁₋₄ alkyl or C₁₋₄ alkoxy;
n is 0, 1 or 2;
each R′ is the same or different and represents halogen, C₁₋₄ alkyl or C₁₋₄ alkoxy; and
m is 0, 1 or 2.

24. A quinazoline derivative according to claim 1 having formula (Ic), or a pharmaceutically acceptable salt thereof: 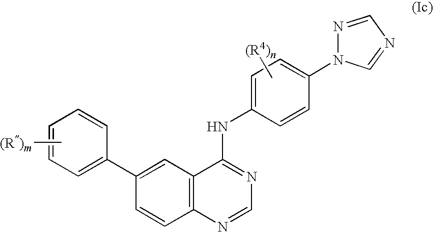
wherein:
each R⁴ is the same or different and represents halogen, C₁₋₄ alkyl or C₁₋₄ alkoxy;
n is 0, 1 or 2;
m is 1 or 2; and
R″ is -A′, -Het-A′, -L-A′, -Het-L-A′, -L-Het-A′, -Het-L-Het′-A′ or -Het-L-Het′-L′, wherein A′, Het, L, Het′ and L′ are as defined in claim 1.

25. (canceled)

26. A pharmaceutical composition which comprises a quinazoline derivative of the formula (I), as defined in claim 1, or a pharmaceutically acceptable salt thereof, and a pharmaceutically acceptable carrier or diluent.

27-39. (canceled)

40. A pharmaceutical composition, comprising:
(a) a quinazoline derivative of the formula (I), as defined in claim 1, or a pharmaceutically acceptable salt thereof; and
(b) interferon or an interferon derivative and/or ribavirin or a ribavirin derivative;
and a pharmaceutically acceptable carrier or diluent.

41-43. (canceled)

44. A method of treating a patient suffering from or susceptible to a flaviviridae infection, which method comprises administering to said patient an effective amount of a quinazoline derivative of the formula (I) as defined in claim 1 or a pharmaceutically acceptable salt thereof.

45. The method according to claim 44, wherein the flavivirdae infection is a hepacivirus infection.

46. The method according to claim 45, wherein the hepacivirus infection is an infection by a hepatitis C virus.

47. The method according to claim 46, further comprising the step of administering to said patient (a) interferon or a derivative thereof and/or (b) ribavirin or a derivative thereof.
